(19) United States
(12) Patent Application Publication
(10) Pub. No.: US 2016/0151201 A1
(43) Pub. Date: Jun. 2, 2016

(54) METHODS AND DEVICES FOR SELECTIVE BRAIN COOLING

(71) Applicant: NEUROSAVE, INC., San Francisco, CA (US)

(72) Inventors: Thomas Kreck, San Francisco, CA (US); Seth RODGERS, San Francisco, CA (US); Andrew SCOTT, San Francisco, CA (US)

(73) Assignee: NEUROSAVE, INC., San Francisco, CA (US)

(21) Appl. No.: 14/900,554

(22) PCT Filed: Jun. 20, 2014
(86) PCT No.: PCT/US2014/043509

§ 371 Date: Dec. 21, 2015

Related U.S. Application Data
(60) Provisional application No. 61/837,873, filed on Jun. 21, 2013.

(51) Int. Cl. A61F 7/12

(52) U.S. Cl. CPC A61F 7/12; A61F 2007/0006

(57) ABSTRACT

Cooling devices and method, such as those, for example, configured to cool the brain of a subject.

### CROSS-REFERENCE TO RELATED APPLICATIONS

This application claims priority to U.S. Provisional Patent Application No. 61/837,873, filed Jun. 21, 2013, which is incorporated by reference in its entirety.

### BACKGROUND

1. Field of the Invention

The present invention relates generally to cooling devices and method, and more particularly, but not by way of limitation, to cooling devices and methods configured to cool the brain of a subject.

2. Description of Related Art

Brain injury is common, devastating, and often expensive to treat. Management of a subject's brain temperature has been recommended by the American Heart Association (AHA) as the standard of care for survivors of cardiac arrest. Brain temperature management also has been used to treat birth-related cerebral damage. Brain temperature management has been studied as a method that reverses and prevents fever after stroke and traumatic brain injury. In addition to its use after brain injury, brain temperature management has been used for more than 50 years to prevent brain injury during cardiac, vascular, and neurological surgery. Brain temperature management is relevant to a variety of central nervous system conditions, including stroke, mechanical brain trauma, and spinal cord injury. A variety of devices have been proposed for therapeutic organ cooling and, in particular, therapeutic cooling of the brain. Such devices generally fall into one of two broad categories: systemic devices and selective devices.

Systemic devices are widely used today, but limitations, such as systemic toxicity caused by cooling the body core and delays in reaching desired brain temperature, diminish the benefit that patients may receive. On the other hand, selective cooling enables, for example, the creation of a temperature gradient between the brain and the body core. Selective cooling also can reduce complications associated with body core cooling, improve patient safety, and enable deep cooling of the brain tissue to achieve neuro-protection.

In general, a high degree of selectivity in temperature management has required a high, and generally undesirable, degree of invasiveness. Surgically invasive devices, such as intravascular devices, often focus on cooling the blood supply to a target area and warming the returning blood supply to prevent cooling of the body core. Intravascular systems and other similarly invasive devices, however, may not be suitable for rapid deployment because, for example, they may require intervention by a surgeon. A further limitation of catheter-based devices is that they require surgical invasion of a major blood vessel, introducing risk of infection, bleeding, thrombosis, rupture of the blood vessel, dissection of the blood vessel wall, and introduction or dislodging debris in the vasculature. These risks are further increased when an intravascular warming catheter is introduced to re-warm blood flow returning from the cooled organ(s).

Other selective, brain-focused, non-invasive cooling devices require nebulized fluids that undergo a phase change (evaporation) to maximize a rate of heat transfer from the body. An example of this method is described, for example, in U.S. Pat. No. 7,837,722 to Barbut et al. Drawbacks of this approach include exposure of the patient to fluorocarbon coolant (if used as a free flowing liquid), exposure of bystanders to fluorocarbon coolant, and the formation of entrained debris that is difficult to recapture as the coolant leaves the patient. Also, this approach appears to yield a relatively slow cooling rate in human trials and a shallow average depth of cooling of steady state reduction in brain temperature.

Another selective cooling device is described in U.S. Pat. No. 7,189,253 to Lunderqvist et al. The Lunderqvist devices introduce fluid filled balloons into the nasal cavity and cool the cavity by recirculating cold fluid. These devices affect brain temperature by adjusting the temperature of the cooling fluid based on measurement of tympanic membrane temperature. Drawbacks of this approach include a reduction in heat transfer rate due to a reduction in surface area exploited (e.g., the surface area of the sinuses is excluded and the air in the sinuses acts as a barrier to heat transfer) and the heat transfer resistance of the balloon itself.

Other conventional approaches utilize balloon-based devices, such as those disclosed by Takeda in U.S. Patent Publication Nos. 2008/0086186 and 2009/0177258. Such contained use of fluids generally does not, for example, provide good surface contact with the tissues of the airway, reducing heat transfer.

### SUMMARY

This disclosure includes embodiments of cooling devices and methods configured to cool the brain of a subject.

Some embodiments of the present cooling devices include a pump; a first blockage configured to be inserted through a nostril of a subject and disposed in the caudal region of the nasal cavity such that resistance to cooling fluid flow in the caudal region of the nasal cavity is increased; and a first tube configured to be inserted through a nostril of a subject and directed toward the cranial region of the nasal cavity, the first tube coupled to the pump such that the pump can pump fluid through the first tube and into the nasal cavity; where the device is configured to pump fluid into the nasal cavity of the subject to cool the brain of the subject. In some embodiments, the device is configured to cool the brain of the subject until a brain to body temperature gradient of at least 1° C. is achieved (e.g., a temperature gradient of 1° C., 2° C., 3° C., 4° C., 5° C., 6° C., 7° C., 8° C., 9° C., 10° C., or more). In some embodiments, the device is configured to cool the brain of the subject with fluid that is cooler than room temperature (e.g., cooler than 28° C., 26° C., 24° C., 22° C., 20° C., 18° C., or cooler). In some embodiments, the device is configured to cool the brain of the subject for at least one hour (e.g., 1 hour, 2 hours, 3 hours, 4 hours, 5 hours, 6 hours, 7 hours, 8 hours, 9 hours, 10 hour, or more). In some embodiments, the device is configured to cool the brain of the subject with at least 250 milliliters of fluid (e.g., 250 milliliters, 500 milliliters, 750 milliliters, 1 liter, 1.25 liters, 1.5 liters, 1.75 liters, 2 liters, or more).

Some embodiments of the present devices comprise a second blockage configured to be inserted through a nostril of a subject and disposed in the caudal region of the nasal cavity such that resistance to cooling fluid flow in the caudal region of the nasal cavity is increased. Some embodiments comprise a second tube configured to be inserted through a nostril of a subject and directed toward the cranial region of the nasal cavity, the second tube coupled to the pump such that the pump can pump fluid through the second tube and into the nasal cavity. In some embodiments, the first blockage is disposed between the septum and an inferior nasal turbinate. In some embodiments, the first blockage and the second blockage is each disposed between the septum and an inferior nasal turbinate. In some embodiments, the second blockage is disposed between the septum and a middle nasal turbinate. In some embodiments, the first blockage extends to the choana. In some embodiments, the second blockage extends to the choana. In some embodiments, after the first tube is inserted through a nostril of a subject and directed toward the cranial region of the nasal cavity, the first tube is directed toward the superior nasal turbinate. In some embodiments, after the second tube is inserted through a nostril of a subject and directed toward the cranial region of the nasal cavity, the second tube is directed toward the superior nasal turbinate. In some embodiments, the first tube comprises a plurality of openings through which fluid can exit the first tube. In some embodiments, the first tube comprises one opening through which fluid can exit the first tube. In some embodiments, the second tube comprises a plurality of openings through which fluid can exit the second tube. In some embodiments, the second tube comprises one opening through which fluid can exit the second tube.

In some embodiments of the present cooling devices, the first blockage comprises an inflatable cuff that is positioned such that, after the first blockage is inserted through a nostril of a subject, the inflatable cuff can be inflated to further increase resistance to cooling fluid flow in the caudal region of the nasal cavity. In some embodiments, the inflatable cuff can further increase resistance to cooling fluid flow in the caudal region of the nasal cavity by applying pressure to at least one of the septum, a middle turbinate, and an inferior turbinate. In some embodiments, the first blockage and the first tube are each coupled to an inflatable cuff such that, after the first blockage and the first tube are each inserted through a nostril of a subject, the inflatable cuff can be inflated to further increase resistance to cooling fluid flow in the caudal region of the nasal cavity. In some embodiments, the inflatable cuff can further increase resistance to cooling fluid flow in the caudal region of the nasal cavity by applying pressure to at least one of the septum, a middle turbinate, and an inferior turbinate. In some embodiments, the second blockage comprises an inflatable cuff that is positioned such that, after the second blockage is inserted through a nostril of a subject, the inflatable cuff can be inflated to further increase resistance to cooling fluid flow in the caudal region of the nasal cavity. In some embodiments, the inflatable cuff can further increase resistance to cooling fluid flow in the caudal region of the nasal cavity by applying pressure to at least one of the septum, a middle turbinate, and an inferior turbinate. In some embodiments, the second blockage and the second tube are each coupled to an inflatable cuff such that, after the second blockage and the second tube are each inserted through a nostril of a subject, the inflatable cuff can be inflated to further increase resistance to cooling fluid flow in the caudal region of the nasal cavity. In some embodiments, the inflatable cuff can further increase resistance to cooling fluid flow in the caudal region of the nasal cavity by applying pressure to at least one of the septum, a middle turbinate, and an inferior turbinate. In some embodiments, the first blockage and the first tube are coupled to an inflatable cuff and the second blockage and the second tube are coupled to an inflatable cuff such that after the first blockage, the first tube, the second blockage, and the second tube are inserted through a nostril of a subject, the inflatable cuffs can be inflated to further increase resistance to cooling fluid flow in the caudal region of the nasal cavity. In some embodiments, the inflatable cuffs can further increase resistance to cooling fluid flow in the caudal region of the nasal cavity by applying pressure to at least one of the septum, a middle turbinate, and an inferior turbinate.

Some embodiments of the present devices comprise a sphenoid sinus tube coupled to the pump and configured to be inserted through a nostril of a subject and into a sphenoid sinus of the subject such that the pump can pump fluid through the sphenoid sinus tube and into the sphenoid sinus. Some embodiments of the present devices comprise a third blockage configured to be inserted through a nostril of a subject and disposed in the caudal region of the nasal cavity such that resistance to cooling fluid flow in the caudal region of the nasal cavity is increased; and a fourth blockage configured to be inserted through a nostril of a subject and disposed in the caudal region of the nasal cavity such that resistance to cooling fluid flow in the caudal region of the nasal cavity is increased; where the third blockage and the fourth blockage is each disposed between the septum and a middle nasal turbinate. In some embodiments, a portion of at least one of the first blockage, the second blockage, the third blockage, and the fourth blockage is substantially semi-cylindrical. In some embodiments, the portion of at least one of the first blockage, the second blockage, the third blockage, and the fourth blockage that is substantially semi-cylindrical is the portion that is in contact with the respective turbinate. In some embodiments, a portion of at least one of the first blockage and the second blockage is substantially semi-cylindrical. In some embodiments, the portion of at least one of the first blockage and the second blockage that is substantially semi-cylindrical is the portion that is in contact with the respective turbinate. In some embodiments, the pump comprises at least a first pump and a second pump, and the first tube is coupled to the first pump and the second tube is coupled to the second pump. In some embodiments, the first tube and the second tube each comprises a pressure sensor. In some embodiments, the third blockage extends to the choana. In some embodiments, the fourth blockage extends to the choana. In some embodiments, the third blockage can comprise an inflatable cuff that is positioned such that, after the third blockage is inserted through a nostril of a subject, the inflatable cuff can be inflated to further increase resistance to cooling fluid flow in the caudal region of the nasal cavity. In some embodiments, the fourth blockage can comprise an inflatable cuff that is positioned such that, after the fourth blockage is inserted through a nostril of a subject, the inflatable cuff can be inflated to further increase resistance to cooling fluid flow in the caudal region of the nasal cavity. In some embodiments, a blockage can comprise a circle configuration. In some embodiments, a blockage can comprise an oval configuration. In some embodiments, a blockage can comprise a teardrop configuration. In some embodiments, a blockage can comprise a frustum configuration.

Some embodiments of the present cooling devices comprise a pump; and a first sphenoid sinus tube coupled to the pump and configured to be inserted through a nostril of a subject and into a sphenoid sinus of the subject such that the pump can pump fluid through the first sphenoid sinus tube and into the sphenoid sinus; where the device is configured to cool the brain of the subject. In some embodiments, the device is configured to cool the brain of the subject until a brain to body temperature gradient of at least 1° C. is achieved (e.g., a temperature gradient of 1° C., 2° C., 3° C., 4° C., 5° C., 6° C., 7° C., 8° C., 9° C., 10° C., or more). In some embodiments, the device is configured to cool the brain of the subject with fluid that is cooler than room temperature (e.g., cooler than 28° C., 26° C., 24° C., 22° C., 20° C., 18° C., or cooler). In some embodiments, the device is configured to cool the brain of the subject for at least one hour (e.g., 1 hour, 2 hours, 3 hours, 4 hours, 5 hours, 6 hours, 7 hours, 8 hours, 9 hours, 10 hour, or more). In some embodiments, the device is configured to cool the brain of the subject with at least 250 milliliters of fluid (e.g., 250 milliliters, 500 milliliters, 750 milliliters, 1 liter, 1.25 liters, 1.5 liters, 1.75 liters, 2 liters, or more).

Some embodiments of the present devices comprise a second sphenoid sinus tube configured to be inserted through a nostril of a subject and into a sphenoid sinus of the subject, where the device is configured to remove fluid from the sphenoid sinus through the second sphenoid sinus tube. In some embodiments, the first sphenoid sinus tube comprises a multi-lumen catheter, where the pump is configured to pump fluid through a first lumen of the first sphenoid sinus tube and into the sphenoid sinus, and where the device is configured to remove fluid from the sphenoid sinus through a second lumen of the first sphenoid sinus tube.

Some embodiments of the present devices comprise a third sphenoid sinus tube coupled to the pump and configured to be inserted through a nostril of a subject and into a sphenoid sinus of the subject such that the pump can pump fluid through the third sphenoid sinus tube and into the sphenoid sinus. Some embodiments of the present devices comprise a fourth sphenoid sinus tube configured to be inserted through a nostril of a subject and into a sphenoid sinus of the subject, where the device is configured to remove fluid from the sphenoid sinus through the fourth sphenoid sinus tube. In some embodiments, the third sphenoid sinus tube comprises a multi-lumen catheter, where the pump is configured to pump fluid through a first lumen of the third sphenoid sinus tube and into the sphenoid sinus, and where the device is configured to remove fluid from the sphenoid sinus through a second lumen of the third sphenoid sinus tube.

In some embodiments, a sphenoid sinus tube comprises a substantially malleable material. In some embodiments, a sphenoid sinus tube comprises a first portion comprising a substantially rigid material and a second portion comprising a substantially malleable material. Some embodiments of the present devices can comprise a stopper coupled to a sphenoid sinus tube and configured to limit the extent to which the sphenoid sinus tube can enter the sphenoid sinus. In some embodiments, a sphenoid sinus tube comprises a friction-reducing coating. In some embodiments, a sphenoid sinus tube comprises a first portion and a second portion comprising a plurality of openings through which a fluid can exit the sphenoid sinus tube. Some embodiments of the present devices comprise a pressure sensor coupled to a sphenoid sinus tube and configured to detect if pressure in the sphenoid sinus meets or exceeds a threshold pressure. In some embodiments, a sphenoid sinus tube comprises a first portion and a second portion configured such that, after the sphenoid sinus tube is inserted into the sphenoid sinus of the subject, the second portion of the sphenoid sinus tube prevents the sphenoid sinus tube from exiting the sphenoid sinus of the subject. In some embodiments, a sphenoid sinus tube comprises a first portion and a second portion configured such that, after the sphenoid sinus tube is inserted into the sphenoid sinus of the subject, the second portion of the sphenoid sinus tube expands to prevent the sphenoid sinus tube from exiting the sphenoid sinus of the subject. In some embodiments, a sphenoid sinus tube comprises a first portion and a second portion configured such that, after the sphenoid sinus tube is inserted into the sphenoid sinus of the subject, the second portion of the first sphenoid sinus tube coils to prevent the sphenoid sinus tube from exiting the sphenoid sinus of the subject. In some embodiments, after a sphenoid sinus tube is inserted into the sphenoid sinus of the subject, the sphenoid sinus tube is coupled to the nose of the patient such that the sphenoid sinus tube is prevented from exiting the sphenoid sinus of the subject. In some embodiments, a sphenoid sinus tube comprises an inflatable cuff positioned such that, after the sphenoid sinus tube is inserted into the sphenoid sinus of the subject, the inflatable cuff can be inflated to engage tissue defining a sphenoid ostium.

In some embodiments, the pump comprises at least a first pump and a second pump, and the first sphenoid sinus tube and the second sphenoid sinus tube are coupled to the first pump, and the third sphenoid sinus tube and the fourth sphenoid sinus tube are coupled to the second pump. In some embodiments, the first sphenoid sinus tube, the second sphenoid sinus tube, the third sphenoid sinus tube, and the fourth sphenoid sinus tube each comprises a pressure sensor. In some embodiments, the pump comprises at least a first pump, a second pump, a third pump, and a fourth pump, and the first sphenoid sinus tube is coupled to the first pump, the second sphenoid sinus tube is coupled to the second pump, the third sphenoid sinus tube is coupled to the third pump, and the fourth sphenoid sinus tube is coupled to the fourth pump. In some embodiments, the first sphenoid sinus tube, the second sphenoid sinus tube, the third sphenoid sinus tube, and the fourth sphenoid sinus tube each comprises a pressure sensor.

Some embodiments of the present devices comprise a pump; a first blockage configured to be inserted through a nostril of a subject and disposed in the caudal region of the nasal cavity such that resistance to cooling fluid flow in the caudal region of the nasal cavity is increased; a first tube configured to be inserted through a nostril of a subject and directed toward the cranial region of the nasal cavity, the first tube coupled to the pump such that the pump can pump fluid through the first tube and into the nasal cavity; and a first sphenoid sinus tube configured to be inserted through a nostril of a subject and into a sphenoid sinus of the subject, the first sphenoid sinus tube coupled to the pump such that the pump can pump fluid through the first sphenoid sinus tube and into the nasal cavity; where the device is configured to cool the brain of the subject. In some embodiments, the device is configured to cool the brain of the subject until a brain to body temperature gradient of at least 1° C. is achieved (e.g., a temperature gradient of 1° C., 2° C., 3° C., 4° C., 5° C., 6° C., 7° C., 8° C., 9° C., 10° C., or more). In some embodiments, the device is configured to cool the brain of the subject with fluid that is cooler than room temperature (e.g., cooler than 28° C., 26° C., 24° C., 22° C., 20° C., 18° C., or cooler). In some embodiments, the device is configured to cool the brain of the subject for at least one hour (e.g., 1 hour, 2 hours, 3 hours, 4 hours, 5 hours, 6 hours, 7 hours, 8 hours, 9 hours, 10 hour, or more). In some embodiments, the device is configured to cool the brain of the subject with at least 250 milliliters of fluid (e.g., 250 milliliters, 500 milliliters, 750 milliliters, 1 liter, 1.25 liters, 1.5 liters, 1.75 liters, 2 liters, or more).

Some embodiments of the present devices comprise a pump and a first sphenoid sinus tube coupled to the pump and configured to be inserted through a nostril of a subject and into a sphenoid sinus of the subject such that the pump can remove fluid from the sphenoid sinus through the first sphenoid sinus tube; where the device is configured to cool the brain of the subject. In some embodiments, the devices further comprise a second sphenoid sinus tube coupled to the pump and configured to be inserted through a nostril of a subject and into a sphenoid sinus of the subject such that the pump can remove fluid from the sphenoid sinus through the third sphenoid sinus tube. In some embodiments, the pump comprises at least a first pump and a second pump, and the first sphenoid sinus tube is coupled to the first pump, and the second sphenoid sinus tube is coupled to the second pump. In some embodiments, the first sphenoid sinus tube and the second sphenoid sinus tube each comprises a pressure sensor.

Some embodiments of the present devices comprise a second blockage configured to be inserted through a nostril of a subject and disposed in the caudal region of the nasal cavity such that resistance to cooling fluid flow in the caudal region of the nasal cavity is increased. Some embodiments comprise a second tube configured to be inserted through a nostril of a subject and directed toward the cranial region of the nasal cavity, the second tube coupled to the pump such that the pump can pump fluid through the second tube and into the nasal cavity. In some embodiments, the first blockage is disposed between the septum and an inferior nasal turbinate. In some embodiments, the first blockage and the second blockage is each disposed between the septum and an inferior nasal turbinate. In some embodiments, the second blockage is disposed between the septum and a middle nasal turbinate. In some embodiments, the first blockage extends to the choana. In some embodiments, the second blockage extends to the choana. In some embodiments, after the first tube is inserted through a nostril of a subject and directed toward the cranial region of the nasal cavity, the first tube is directed toward the superior nasal turbinate. In some embodiments, after the second tube is inserted through a nostril of a subject and directed toward the cranial region of the nasal cavity, the second tube is directed toward the superior nasal turbinate. In some embodiments, the first tube comprises a plurality of openings through which fluid can exit the first tube. In some embodiments, the first tube comprises one opening through which fluid can exit the first tube. In some embodiments, the second tube comprises a plurality of openings through which fluid can exit the second tube. In some embodiments, the second tube comprises one opening through which fluid can exit the second tube.

In some embodiments of the present cooling devices, the first blockage comprises an inflatable cuff that is positioned such that, after the first blockage is inserted through a nostril of a subject, the inflatable cuff can be inflated to further increase resistance to cooling fluid flow in the caudal region of the nasal cavity. In some embodiments, the inflatable cuff can further increase resistance to cooling fluid flow in the caudal region of the nasal cavity by applying pressure to at least one of the septum, a middle turbinate, and an inferior turbinate. In some embodiments, the first blockage and the first tube are each coupled to an inflatable cuff such that, after the first blockage and the first tube are each inserted through a nostril of a subject, the inflatable cuff can be inflated to further increase resistance to cooling fluid flow in the caudal region of the nasal cavity. In some embodiments, the inflatable cuff can further increase resistance to cooling fluid flow in the caudal region of the nasal cavity by applying pressure to at least one of the septum, a middle turbinate, and an inferior turbinate. In some embodiments, the second blockage comprises an inflatable cuff that is positioned such that, after the second blockage is inserted through a nostril of a subject, the inflatable cuff can be inflated to further increase resistance to cooling fluid flow in the caudal region of the nasal cavity. In some embodiments, the inflatable cuff can further increase resistance to cooling fluid flow in the caudal region of the nasal cavity by applying pressure to at least one of the septum, a middle turbinate, and an inferior turbinate. In some embodiments, the second blockage and the second tube are each coupled to an inflatable cuff such that, after the second blockage and the second tube are each inserted through a nostril of a subject, the inflatable cuff can be inflated to further increase resistance to cooling fluid flow in the caudal region of the nasal cavity. In some embodiments, the inflatable cuff can further increase resistance to cooling fluid flow in the caudal region of the nasal cavity by applying pressure to at least one of the septum, a middle turbinate, and an inferior turbinate. In some embodiments, the first blockage and the first tube are coupled to an inflatable cuff and the second blockage and the second tube are coupled to an inflatable cuff such that after the first blockage, the first tube, the second blockage, and the second tube are inserted through a nostril of a subject, the inflatable cuffs can be inflated to further increase resistance to cooling fluid flow in the caudal region of the nasal cavity. In some embodiments, the inflatable cuffs can further increase resistance to cooling fluid flow in the caudal region of the nasal cavity by applying pressure to at least one of the septum, a middle turbinate, and an inferior turbinate.

Some embodiments of the present devices comprise a sphenoid sinus tube coupled to the pump and configured to be inserted through a nostril of a subject and into a sphenoid sinus of the subject such that the pump can pump fluid through the sphenoid sinus tube and into the sphenoid sinus. Some embodiments of the present devices comprise a third blockage configured to be inserted through a nostril of a subject and disposed in the caudal region of the nasal cavity such that resistance to cooling fluid flow in the caudal region of the nasal cavity is increased; and a fourth blockage configured to be inserted through a nostril of a subject and disposed in the caudal region of the nasal cavity such that resistance to cooling fluid flow in the caudal region of the nasal cavity is increased; where the third blockage and the fourth blockage is each disposed between the septum and a middle nasal turbinate. In some embodiments, a portion of at least one of the first blockage, the second blockage, the third blockage, and the fourth blockage is substantially semi-cylindrical. In some embodiments, the portion of at least one of the first blockage, the second blockage, the third blockage, and the fourth blockage that is substantially semi-cylindrical is the portion that is in contact with the respective turbinate. In some embodiments, a portion of at least one of the first blockage and the second blockage is substantially semi-cylindrical. In some embodiments, the portion of at least one of the first blockage and the second blockage that is substantially semi-cylindrical is the portion that is in contact with the respective turbinate. In some embodiments, the third blockage extends to the choana. In some embodiments, the fourth blockage extends to the choana. In some embodiments, the third blockage can comprise an inflatable cuff that is positioned such that, after the third blockage is inserted through a nostril of a subject, the inflatable cuff can be inflated to further increase resistance to cooling fluid flow in the caudal region of the nasal cavity. In some embodiments, the fourth blockage can comprise an inflatable cuff that is positioned such that, after the fourth blockage is inserted through a nostril of a subject, the inflatable cuff can be inflated to further increase resistance to cooling fluid flow in the caudal region of the nasal cavity. In some embodiments, a blockage can comprise a circle configuration. In some embodiments, a blockage can comprise an oval configuration. In some embodiments, a blockage can comprise a teardrop configuration. In some embodiments, a blockage can comprise a frustum configuration.

Some embodiments of the present devices comprise a second sphenoid sinus tube configured to be inserted through a nostril of a subject and into a sphenoid sinus of the subject, where the device is configured to remove fluid from the sphenoid sinus through the second sphenoid sinus tube. In some embodiments, the first sphenoid sinus tube comprises a multi-lumen catheter, where the pump is configured to pump fluid through a first lumen of the first sphenoid sinus tube and into the sphenoid sinus, and where the device is configured to remove fluid from the sphenoid sinus through a second lumen of the first sphenoid sinus tube.

Some embodiments of the present devices comprise a third sphenoid sinus tube coupled to the pump and configured to be inserted through a nostril of a subject and into a sphenoid sinus of the subject such that the pump can pump fluid through the third sphenoid sinus tube and into the sphenoid sinus. Some embodiments of the present devices comprise a fourth sphenoid sinus tube configured to be inserted through a nostril of a subject and into a sphenoid sinus of the subject, where the device is configured to remove fluid from the sphenoid sinus through the fourth sphenoid sinus tube. In some embodiments, the third sphenoid sinus tube comprises a multi-lumen catheter, where the pump is configured to pump fluid through a first lumen of the third sphenoid sinus tube and into the sphenoid sinus, and where the device is configured to remove fluid from the sphenoid sinus through a second lumen of the third sphenoid sinus tube.

In some embodiments, a sphenoid sinus tube comprises a substantially malleable material. In some embodiments, a sphenoid sinus tube comprises a first portion comprising a substantially rigid material and a second portion comprising a substantially malleable material. Some embodiments of the present devices can comprise a stopper coupled to a sphenoid sinus tube and configured to limit the extent to which the sphenoid sinus tube can enter the sphenoid sinus. In some embodiments, a sphenoid sinus tube comprises a friction-reducing coating. In some embodiments, a sphenoid sinus tube comprises a first portion and a second portion comprising a plurality of openings through which a fluid can exit the sphenoid sinus tube. Some embodiments of the present devices comprise a pressure sensor coupled to a sphenoid sinus tube and configured to detect if pressure in the sphenoid sinus meets or exceeds a threshold pressure. In some embodiments, a sphenoid sinus tube comprises a first portion and a second portion configured such that, after the sphenoid sinus tube is inserted into the sphenoid sinus of the subject, the second portion of the sphenoid sinus tube prevents the sphenoid sinus tube from exiting the sphenoid sinus of the subject. In some embodiments, a sphenoid sinus tube comprises a first portion and a second portion configured such that, after the sphenoid sinus tube is inserted into the sphenoid sinus of the subject, the second portion of the sphenoid sinus tube expands to prevent the sphenoid sinus tube from exiting the sphenoid sinus of the subject. In some embodiments, a sphenoid sinus tube comprises a first portion and a second portion configured such that, after the sphenoid sinus tube is inserted into the sphenoid sinus of the subject, the second portion of the first sphenoid sinus tube coils to prevent the sphenoid sinus tube from exiting the sphenoid sinus of the subject. In some embodiments, after a sphenoid sinus tube is inserted into the sphenoid sinus of the subject, the sphenoid sinus tube is coupled to the nose of the patient such that the sphenoid sinus tube is prevented from exiting the sphenoid sinus of the subject. In some embodiments, a sphenoid sinus tube comprises an inflatable cuff positioned such that, after the sphenoid sinus tube is inserted into the sphenoid sinus of the subject, the inflatable cuff can be inflated to engage tissue defining a sphenoid ostium. In some embodiments, the pump comprises at least a first pump and a second pump, and the first tube is coupled to the first pump, and the second tube is coupled to the second pump. In some embodiments, the first tube and the second tube each comprises a pressure sensor. In some embodiments, the pump comprises at least a first pump and a second pump, and the first sphenoid sinus tube and the second sphenoid sinus tube are coupled to the first pump, and the third sphenoid sinus tube and the fourth sphenoid sinus tube are coupled to the second pump. In some embodiments, the first sphenoid sinus tube, the second sphenoid sinus tube, the third sphenoid sinus tube, and the fourth sphenoid sinus tube each comprises a pressure sensor. In some embodiments, the pump comprises at least a first pump, a second pump, a third pump, and a fourth pump, and the first sphenoid sinus tube is coupled to the first pump, the second sphenoid sinus tube is coupled to the second pump, the third sphenoid sinus tube is coupled to the third pump, and the fourth sphenoid sinus tube is coupled to the fourth pump. In some embodiments, the first sphenoid sinus tube, the second sphenoid sinus tube, the third sphenoid sinus tube, and the fourth sphenoid sinus tube each comprises a pressure sensor.

Some embodiments of the present devices comprise a pump; and a first nasal insert coupled to the pump such that the pump can pump fluid through the first nasal insert, the first nasal insert comprising a first portion configured to be inserted through a nostril of a subject and directed toward the cranial region of the nasal cavity; and a second portion configured to be inserted through a nostril of a subject and disposed in the caudal region of the nasal cavity such that resistance to cooling fluid flow in the caudal region of the nasal cavity is increased; where the device is configured to pump fluid into the nasal cavity of the subject to cool the brain of the subject. In some embodiments, the device is configured to cool the brain of the subject until a brain to body temperature gradient of at least 1° C. is achieved (e.g., a temperature gradient of 1° C., 2° C., 3° C., 4° C., 5° C., 6° C., 7° C., 8° C., 9° C., 10° C., or more). In some embodiments, the device is configured to cool the brain of the subject with fluid that is cooler than room temperature (e.g., cooler than 28° C., 26° C., 24° C., 22° C., 20° C., 18° C., or cooler). In some embodiments, the device is configured to cool the brain of the subject for at least one hour (e.g., 1 hour, 2 hours, 3 hours, 4 hours, 5 hours, 6 hours, 7 hours, 8 hours, 9 hours, 10 hour, or more). In some embodiments, the device is configured to cool the brain of the subject with at least 250 milliliters of fluid (e.g., 250 milliliters, 500 milliliters, 750 milliliters, 1 liter, 1.25 liters, 1.5 liters, 1.75 liters, 2 liters, or more).

Some embodiments of the present devices comprise a second nasal insert coupled to the pump such that the pump can pump fluid through the second nasal insert, the second nasal insert comprising a first portion configured to be inserted through a nostril of a subject and directed toward the cranial region of the nasal cavity; and a second portion configured to be inserted through a nostril of a subject and disposed in the caudal region of the nasal cavity such that resistance to cooling fluid flow in the caudal region of the nasal cavity is increased; where the device is configured to pump fluid into the nasal cavity of the subject to cool the brain of the subject. In some embodiments, the second portion of the first nasal insert is configured to be disposed between the septum and an inferior nasal turbinate of the subject. In some embodiments, the second portion of the first nasal insert and the second portion of the second nasal insert is each configured to be disposed between the septum and an inferior nasal turbinate of the subject. In some embodiments, the second portion of the first nasal insert is configured to be disposed between the septum and a middle nasal turbinate of the subject. In some embodiments, the second portion of the first nasal insert and the second portion of the second nasal insert is each configured to be disposed between the septum and a middle nasal turbinate of the subject. In some embodiments, a nasal insert can comprise a circle configuration. In some embodiments, a nasal insert can comprise an oval configuration. In some embodiments, a nasal insert can comprise a teardrop configuration. In some embodiments, a nasal insert can comprise a frustum configuration. In some embodiments, a second portion of a nasal insert can comprise a plurality of openings, and the second portion of the nasal insert can be positioned such that, if fluid moves through the second portion of the nasal insert, fluid can exit the second portion of the nasal insert through the plurality of openings and toward the cranial region of the nasal cavity. In some embodiments, the pump comprises at least a first pump and a second pump, and the first nasal insert is coupled to the first pump, and the second nasal insert is coupled to the second pump. In some embodiments, the first nasal insert and the second nasal insert each comprises a pressure sensor.

Some embodiments of the present devices comprise: a multi-lumen catheter configured to be inserted through the mouth of a subject and into the trachea, the multi-lumen catheter comprising: a first lumen configured such that, if the multi-lumen catheter is inserted into the trachea of a subject, the first lumen is in fluid communication with the lungs of the subject; and a second lumen; a first inflatable cuff configured to be inserted through the mouth of a subject and into the trachea, the first inflatable cuff configured such that, if the first inflatable cuff is inserted into the trachea of a subject and inflated, the first inflatable cuff substantially prevents fluid from entering the lungs of the subject; and a second inflatable cuff configured to be inserted through the mouth of a subject and into the trachea distal to the first inflatable cuff, the second inflatable cuff configured such that, if the second inflatable cuff is inserted into the trachea of a subject and inflated, the second inflatable cuff substantially prevents fluid from entering the lungs of the subject; where the first inflatable cuff and the second inflatable cuff define three regions: a first region proximal to the first inflatable cuff; a second region distal to the second inflatable cuff; and a buffer region between the first inflatable cuff and the second inflatable cuff, and where the second lumen of the multi-lumen catheter is in fluid communication with the buffer region such that pressure in the buffer region can be increased and decreased. In some embodiments, at least one of the first inflatable cuff and the second inflatable cuff is coupled to the multi-lumen catheter. In some embodiments, at least one of the first inflatable cuff and the second inflatable cuff is unitary with the multi-lumen catheter. In some embodiments, the device, if activated, is configured to increase pressure in the buffer region to be equal to or greater than pressure in the first region at the first inflatable cuff. In some embodiments, the device, if activated, is configured to increase pressure in the buffer region to at least 20 cmH₂O. In some embodiments, the device further comprises a first sensor configured to detect at least one of pressure, temperature, and impedance in the first region. In some embodiments, the device comprises a second sensor configured to detect pressure in the buffer region. In some embodiments, the device comprises a third sensor configured to detect at least one of pressure, temperature, and impedance in the second region. In some embodiments, the multi-lumen catheter further comprises a third lumen in fluid communication with the first region such that pressure in the first region can be increased and decreased. In some embodiments, the device further comprises a cuff sensor disposed interior to at least one of the first inflatable cuff and the second inflatable cuff, the cuff sensor configured to detect if pressure in the at least one of the first inflatable cuff and the second inflatable cuff increases or decreases. In some embodiments, the cuff sensor comprises one of a temperature sensor and an impedance sensor. In some embodiments, the device further comprises a sump line disposed in the second lumen of the multi-lumen catheter such that the sump line is in fluid communication with the buffer region, where the device, if activated, is configured to remove fluid from the buffer region through the sump line. In some embodiments, a first end of the sump line comprises a bevel cut. In some embodiments, a sump sensor is disposed at a first end of the sump line, and the device is configured to activate (e.g., pump) if the sump sensor detects fluid in the buffer region.

Some embodiments of the present devices comprise a pump; and a first tube configured to be disposed adjacent to a nostril of a subject such that fluid is directed into the nose and is substantially prevented from exiting the nose, the first tube coupled to the pump such that the pump can pump fluid through the first tube and into the nose; where the device is configured to pump fluid into the nasal cavity of the subject to cool the brain of the subject. In some embodiments, the present devices comprise a first sphenoid sinus tube coupled to the pump and configured to be inserted through a nostril of a subject and into a sphenoid sinus of the subject such that the pump can pump fluid through the first sphenoid sinus tube and into the sphenoid sinus. In some embodiments, the first tube and the first sphenoid sinus tube are substantially coaxial. In some embodiments, the present devices comprise a first sphenoid sinus tube coupled to the pump and configured to be inserted through a nostril of a subject and into a sphenoid sinus of the subject such that the pump can remove fluid from the sphenoid sinus through the first sphenoid sinus tube. In some embodiments, the devices comprise a second sphenoid sinus tube coupled to the pump and configured to be inserted through a nostril of a subject and into a sphenoid sinus of the subject such that the pump can pump fluid through the second sphenoid sinus tube and into the sphenoid sinus. In some embodiments, the devices comprise a second sphenoid sinus tube coupled to the pump and configured to be inserted through a nostril of a subject and into a sphenoid sinus of the subject such that the pump can remove fluid from the sphenoid sinus through the second sphenoid sinus tube. In some embodiments, the devices comprise a second tube configured to be disposed adjacent to a nostril of a subject such that fluid is directed into the nose and is substantially prevented from exiting the nose, the second tube coupled to the pump such that the pump can pump fluid through the second tube and into the nose. In some embodiments, the pump comprises at least a first pump and a second pump, and the first tube is coupled to the first pump, and the second tube is coupled to the second pump. In some embodiments, the first tube and the second tube each comprises a pressure sensor. In some embodiments, the pump comprises at least a first pump and a second pump, and the first sphenoid sinus tube is coupled to the first pump, and the second sphenoid sinus tube is coupled to the second pump. In some embodiments, the first sphenoid sinus tube and the second sphenoid sinus tube each comprises a pressure sensor. In some embodiments, the pump comprises at least a first pump and a second pump, and the first sphenoid sinus tube is coupled to the first pump, and the second sphenoid sinus tube is coupled to the second pump. In some embodiments, the first sphenoid sinus tube and the second sphenoid sinus tube each comprises a pressure sensor.

Some embodiments of the present devices comprise a pump; a first tube configured to be inserted into a nostril of a subject and configured to terminate prior to the nasal valve such that fluid is directed into the nose, the first tube coupled to the pump such that the pump can pump fluid through the first tube and into the nose; and a stopper configured to substantially prevent fluid from exiting the nose; where the device is configured to pump fluid into the nasal cavity of the subject to cool the brain of the subject. In some embodiments, the devices comprise a second tube configured to be inserted into a nostril of a subject and configured to terminate prior to the nasal valve such that fluid is directed into the nose, the second tube coupled to the pump such that the pump can pump fluid through the second tube and into the nose. In some embodiments, the pump comprises at least a first pump and a second pump, and the first tube is coupled to the first pump, and the second tube is coupled to the second pump. In some embodiments, the first tube and the second tube each comprises a pressure sensor. In some embodiments, the present devices comprise a first sphenoid sinus tube coupled to the pump and configured to be inserted through a nostril of a subject and into a sphenoid sinus of the subject such that the pump can pump fluid through the first sphenoid sinus tube and into the sphenoid sinus. In some embodiments, the present devices comprise a first sphenoid sinus tube coupled to the pump and configured to be inserted through a nostril of a subject and into a sphenoid sinus of the subject such that the pump can remove fluid from the sphenoid sinus through the first sphenoid sinus tube. In some embodiments, the devices comprise a second sphenoid sinus tube coupled to the pump and configured to be inserted through a nostril of a subject and into a sphenoid sinus of the subject such that the pump can pump fluid through the second sphenoid sinus tube and into the sphenoid sinus. In some embodiments, the devices comprise a second sphenoid sinus tube coupled to the pump and configured to be inserted through a nostril of a subject and into a sphenoid sinus of the subject such that the pump can remove fluid from the sphenoid sinus through the second sphenoid sinus tube. In some embodiments, the pump comprises at least a first pump and a second pump, and the first sphenoid sinus tube is coupled to the first pump, and the second sphenoid sinus tube is coupled to the second pump. In some embodiments, the first sphenoid sinus tube and the second sphenoid sinus tube each comprises a pressure sensor. In some embodiments, the first sphenoid sinus tube and the second sphenoid sinus tube each comprises a pressure sensor.

Some embodiments of the present devices comprise a pump and a nasal mask configured to be placed about the nose of a subject such that fluid can enter the nostrils, the nasal mask coupled to the pump such that the pump can pump fluid into the nasal mask and into the nose of the subject; where the device is configured to pump fluid into the nasal cavity of the subject to cool the brain of the subject.

Some embodiments of the present cooling methods comprise inserting a first guide-wire through a nostril of a subject and into a sphenoid sinus of the subject; inserting a first tube over the first guide-wire and into the sphenoid sinus; removing the first guide-wire; and circulating fluid through the first tube and into the sphenoid sinus to cool the brain of the subject. Some embodiments comprise circulating fluid through the first tube and into the sphenoid sinus until a brain to body temperature gradient of at least 1° C. is achieved (e.g., a temperature gradient of 1° C., 2° C., 3° C., 4° C., 5° C., 6° C., 7° C., 8° C., 9° C., 10° C., or more). In some embodiments, the fluid is cooler than room temperature (e.g., cooler than 28° C., 26° C., 24° C., 22° C., 20° C., 18° C., or cooler). In some embodiments, the fluid is circulated through the first tube for at least one hour (e.g., 1 hour, 2 hours, 3 hours, 4 hours, 5 hours, 6 hours, 7 hours, 8 hours, 9 hours, 10 hour, or more). In some embodiments, at least 250 milliliters of fluid is circulated through the first tube (e.g., 250 milliliters, 500 milliliters, 750 milliliters, 1 liter, 1.25 liters, 1.5 liters, 1.75 liters, 2 liters, or more).

Some embodiments of the present methods comprise inserting a first guide-wire through a nostril of a subject and into a sphenoid sinus of the subject; inserting a first tube over the first guide-wire and into the sphenoid sinus; removing the first guide-wire; circulating fluid through the nasal cavity; and removing fluid from the sphenoid sinus through the first tube to cool the brain of the subject.

Some embodiments further comprise increasing the area of a sphenoid ostium to cool the mucosa of the subject. In some embodiments, increasing the area of a sphenoid ostium comprises inflating an inflatable cuff such that the inflatable cuff engages tissue defining the sphenoid ostium. Some embodiments comprise inserting a second tube through a nostril of a subject and into a sphenoid sinus of the subject; and removing fluid from the sphenoid sinus through the second tube. In some embodiments, the first tube comprises a multi-lumen catheter such that fluid can be circulated through a first lumen of the multi-lumen catheter, the method further comprising removing fluid from the sphenoid sinus through a second lumen of the multi-lumen catheter. In some embodiments, fluid is circulated through the first tube at a rate of 1-500 ml/min.

Some embodiments of the present methods further comprise inserting a third tube through a nostril of a subject and into the sphenoid sinus of the subject; and circulating fluid through the third tube and into the sphenoid sinus to cool the brain of the subject. Some embodiments comprise inserting a fourth tube through a nostril of a subject and into a sphenoid sinus; and removing fluid from the sphenoid sinus through the fourth tube. In some embodiments, the third tube comprises a multi-lumen catheter such that fluid can be circulated through a first lumen of the multi-lumen catheter, the method further comprising removing fluid from the sphenoid sinus through a second lumen of the multi-lumen catheter.

Some embodiments of the present methods further comprise, prior to inserting a first tube over the first guide-wire, inserting an inflatable cuff over the first guide-wire; and inflating the inflatable cuff such that the inflatable cuff engages a sphenoid ostium. Some embodiments further comprise deflating (or partially deflating) the inflatable cuff. Some embodiments further comprise removing the inflatable cuff. In some embodiments, inflating the inflatable cuff comprises inflating the inflatable cuff such that adjacent mucosa, connective tissue, and bone is displaced.

Some embodiments of the present methods further comprise, prior to removing the first guide-wire, inserting a second guide-wire into the sphenoid sinus; inserting an inflatable cuff over the second guide-wire; and inflating the inflatable cuff such that the inflatable cuff engages a sphenoid ostium. Some embodiments further comprise deflating (or partially deflating) the inflatable cuff. Some embodiments further comprise removing the inflatable cuff; and removing the second guide-wire. In some embodiments, inflating the inflatable cuff comprises inflating the inflatable cuff such that adjacent mucosa, connective tissue, and bone is displaced.

Some embodiments comprise inserting a first tube through a nostril of a subject; directing the first tube toward the cranial region of the nasal cavity; at least one of decreasing resistance to cooling fluid flow in the cranial region of the nasal cavity and increasing resistance to cooling fluid flow in the caudal region of the nasal cavity; and circulating fluid through the first tube into the nasal cavity to cool the brain of the subject. Some embodiments comprise circulating fluid through the first tube and into the sphenoid sinus until a brain to body temperature gradient of at least 1° C. is achieved (e.g., a temperature gradient of 1° C., 2° C., 3° C., 4° C., 5° C., 6° C., 7° C., 8° C., 9° C., 10° C., or more). In some embodiments, the fluid is cooler than room temperature (e.g., cooler than 28° C., 26° C., 24° C., 22° C., 20° C., 18° C., or cooler). In some embodiments, the fluid is circulated through the first tube for at least one hour (e.g., 1 hour, 2 hours, 3 hours, 4 hours, 5 hours, 6 hours, 7 hours, 8 hours, 9 hours, 10 hour, or more). In some embodiments, at least 250 milliliters of fluid is circulated through the first tube (e.g., 250 milliliters, 500 milliliters, 750 milliliters, 1 liter, 1.25 liters, 1.5 liters, 1.75 liters, 2 liters, or more).

In some embodiments, increasing resistance to cooling fluid flow in the caudal region of the nasal cavity comprises inserting a first blockage through a nostril of a subject; and disposing the first blockage in the caudal region of the nasal cavity. In some embodiments, the first blockage is disposed between the septum and an inferior nasal turbinate. In some embodiments, increasing resistance to cooling fluid flow in the caudal region of the nasal cavity further comprises inserting a second blockage through a nostril of a subject; and disposing the second blockage in the caudal region of the nasal cavity. In some embodiments, the second blockage is disposed between the septum and an inferior nasal turbinate. In some embodiments, the second blockage is disposed between the septum and a middle nasal turbinate. In some embodiments, increasing resistance to cooling fluid flow in the caudal region of the nasal cavity further comprises inserting a third blockage through a nostril of a subject; and disposing the third blockage in the caudal region of the nasal cavity. In some embodiments, the third blockage is disposed between the septum and a middle nasal turbinate. In some embodiments, increasing resistance to cooling fluid flow in the caudal region of the nasal cavity further comprises inserting a fourth blockage through a nostril of a subject; and disposing the fourth blockage in the caudal region of the nasal cavity. In some embodiments, the fourth blockage is disposed between the septum and a middle nasal turbinate. In some embodiments, at least one of the first blockage and the third blockage comprises an inflatable cuff, the method further comprising inflating the inflatable cuff. In some embodiments, at least one of the second blockage and the fourth blockage comprises an inflatable cuff, the method further comprising inflating the inflatable cuff.

In some embodiments, increasing resistance to cooling fluid flow in the caudal region of the nasal cavity comprises introducing a substance that is configured to increase the volume of tissue in the caudal region of the nasal cavity. In some embodiments, the substance comprises at least one of a vasodilator, an irritant, an allergen, a cholinergic, a particulate, nitroglycerine, and/or an erectile dysfunction therapy. In some embodiments, the substance is introduced systemically. In some embodiments, the substance is introduced to the caudal region of the nasal cavity.

Some embodiments of the present methods comprise disposing a sphenoid sinus tube through a nostril of a subject and into the sphenoid sinus of the subject; and circulating fluid through the sphenoid sinus tube into the sphenoid sinus to cool the brain of the subject. Some embodiments comprise circulating fluid through the sphenoid sinus tube and into the sphenoid sinus until a brain to body temperature gradient of at least 1° C. is achieved (e.g., a temperature gradient of 1° C., 2° C., 3° C., 4° C., 5° C., 6° C., 7° C., 8° C., 9° C., 10° C., or more). In some embodiments, the fluid is cooler than room temperature (e.g., cooler than 28° C., 26° C., 24° C., 22° C., 20° C., 18° C., or cooler). In some embodiments, the fluid is circulated through the sphenoid sinus tube for at least one hour (e.g., 1 hour, 2 hours, 3 hours, 4 hours, 5 hours, 6 hours, 7 hours, 8 hours, 9 hours, 10 hour, or more). In some embodiments, at least 250 milliliters of fluid is circulated through the sphenoid sinus tube (e.g., 250 milliliters, 500 milliliters, 750 milliliters, 1 liter, 1.25 liters, 1.5 liters, 1.75 liters, 2 liters, or more).

In some embodiments, decreasing resistance to cooling fluid flow in the cranial region of the nasal cavity comprises inserting a blockage in one or more cranial channels of the nasal cavity; and removing the blockage from the one or more cranial channels. In some embodiments, the blockage is self-expanding. In some embodiments, decreasing resistance to cooling fluid flow in the cranial region of the nasal cavity comprises inserting an inflatable cuff in one or more cranial channels of the nasal cavity; and inflating the inflatable cuff. Some embodiments further comprise deflating (or partially deflating) the inflatable cuff. Some embodiments further comprise removing the inflatable cuff from the one or more cranial channels in the nasal cavity. In some embodiments, decreasing resistance to cooling fluid flow in the cranial region of the nasal cavity comprises introducing a substance that is configured to decrease the volume of tissue in the cranial region of the nasal cavity. In some embodiments, the substance is introduced systemically. In some embodiments, the substance is introduced to the cranial region of the nasal cavity. In some embodiments, the substance is introduced through the first tube. In some embodiments, the substance comprises at least one of an anticholinergic, an antihistamine, a mast cell inhibitor, and/or a vasoconstrictor (e.g., cocaine, oxymetazoline, phenylephrine, xylometazoline, and naphazoline).

Any embodiment of any of the present cooling devices and methods can consist of or consist essentially of—rather than comprise/include/contain/have—any of the described elements and/or features. Thus, in any of the claims, the term “consisting of” or “consisting essentially of” can be substituted for any of the open-ended linking verbs recited above, in order to change the scope of a given claim from what it would otherwise be using the open-ended linking verb.

The term “coupled” is defined as connected, although not necessarily directly, and not necessarily mechanically. Two items are “couplable” if they can be coupled to each other. Unless the context explicitly requires otherwise, items that are couplable are also decouplable, and vice-versa. One non-limiting way in which a first structure is couplable to a second structure is for the first structure to be configured to be coupled (or configured to be couplable) to the second structure.

The terms “a” and “an” are defined as one or more unless this disclosure explicitly requires otherwise.

The term “substantially” is defined as largely but not necessarily wholly what is specified (and includes what is specified; e.g., substantially 90 degrees includes 90 degrees and substantially parallel includes parallel), as understood by a person of ordinary skill in the art. In any disclosed embodiment, the terms “substantially,” “approximately,” and “about” may be substituted with “within [a percentage] of” what is specified, where the percentage includes 0.1, 1, 5, and 10 percent.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a cooling device, or a component of a cooling device, that “comprises,” “has,” “includes” or “contains” one or more elements or features possesses those one or more elements or features, but is not limited to possessing only those elements or features. Likewise, a cooling method that “comprises,” “has,” “includes” or “contains” one or more steps possesses those one or more steps, but is not limited to possessing only those one or more steps. Additionally, terms such as “first” and “second” are used only to differentiate structures or features, and not to limit the different structures or features to a particular order.

The feature or features of one embodiment may be applied to other embodiments, even though not described or illustrated, unless expressly prohibited by this disclosure or the nature of the embodiments.

Details associated with the embodiments described above and others are presented below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. Some of the figures illustrate some of the described elements using graphical symbols that will be understood by those of ordinary skill in the art.

FIG. 1 depicts a side view of a nasal cavity of a subject and a first embodiment of the present cooling devices that comprises a blockage disposed in the caudal region of the nasal cavity of the subject and a tube directed toward the cranial region of the nasal cavity.

FIG. 2 depicts a side view of a nasal cavity of a subject and a second embodiment of the present cooling devices that comprises two blockages disposed in the caudal region of the nasal cavity of the subject and a tube directed toward the cranial region of the nasal cavity.

FIG. 3 depicts a side view of a nasal cavity of a subject and a third embodiment of the present cooling devices that comprises a tube inserted into a sphenoid sinus of the subject.

FIG. 4 depicts a side view of a nasal cavity of a subject and a fourth embodiment of the present cooling devices that comprises a tube inserted into a sphenoid sinus of the subject, a blockage disposed in the caudal region of the nasal cavity, and a tube directed toward the cranial region of the nasal cavity.

FIG. 5 depicts another embodiment of the tube inserted into a sphenoid sinus of the subject of FIG. 3.

FIG. 6A depicts a side view of a nasal cavity of a subject and a fifth embodiment of the present cooling devices that comprises a nasal insert comprising a first portion directed toward the cranial region of the nasal cavity and a second portion disposed in the caudal region of the nasal cavity.

FIG. 6B depicts another embodiment of the cooling device of FIG. 6A.

FIG. 7A depicts a front view of a nasal cavity of a subject and a sixth embodiment of the present cooling devices that comprises two blockages disposed in the caudal region of the nasal cavity of the subject and two tubes directed toward the cranial region of the nasal cavity.

FIG. 7B depicts a blockage, tube, and/or nasal insert of the present cooling devices having a teardrop configuration.

FIG. 7C depicts a blockage, tube, and/or nasal insert of the present cooling devices having a circle configuration.

FIG. 7D depicts a blockage, tube, and/or nasal insert of the present cooling devices having a frustum configuration.

FIG. 8 depicts a front view of a nasal cavity of a subject and a seventh embodiment of the present cooling devices that comprises four blockages disposed in the caudal region of the nasal cavity of the subject and two tubes directed toward the cranial region of the nasal cavity.

FIG. 9 depicts a front view of a nasal cavity of a subject and an eighth embodiment of the present cooling devices that comprises two tubes inserted into a sphenoid sinus of the subject.

FIG. 10 depicts one embodiment of a device that can be used in cooling the brain of a subject to substantially prevent fluid from entering the lungs of a subject. Cross-section A-A depicts the cross-section of the device at the identified location, which can include a cross-sectional representation of lumens, sensors, and/or sensor connections (e.g., electrical).

FIG. 11 depicts a second embodiment of a device that can be used in cooling the brain of a subject to substantially prevent fluid from entering the lungs of a subject. Cross-section A-A depicts the cross-section of the device at the identified location, which can include a cross-sectional representation of lumens, sensors, and/or sensor connections (e.g., electrical).

FIG. 12 depicts a third embodiment of a device that can be used in cooling the brain of a subject to substantially prevent fluid from entering the lungs of a subject. Cross-section A-A depicts the cross-section of the device at the identified location, which can include a cross-sectional representation of lumens, sensors, and/or sensor connections (e.g., electrical).

FIG. 13 depicts a fourth embodiment of a device that can be used in cooling the brain of a subject to substantially prevent fluid from entering the lungs of a subject. Cross-section A-A depicts the cross-section of the device at the identified location, which can include a cross-sectional representation of lumens, sensors, and/or sensor connections (e.g., electrical).

FIG. 14 depicts a cooling collar that can be wrapped at least partially around the neck of a subject.

FIG. 15 depicts a side view of a nasal cavity of a subject and a tenth embodiment of the present cooling devices that comprises a tube adjacent to a nostril of a subject and a tube inserted into a sphenoid sinus of the subject.

FIG. 16 depicts a side view of a nasal cavity of a subject and a eleventh embodiment of the present cooling devices that comprises a tube inserted into a nostril of a subject.

FIG. 17 depicts a side view of a nasal cavity of a subject and a twelfth embodiment of the present cooling devices that comprises a nasal mask about the nose of a subject.

### DESCRIPTION OF ILLUSTRATIVE EMBODIMENTS

Embodiments of cooling devices are disclosed in U.S. Pat. No. 8,308,787, filed Jul. 23, 2010 (“the '787 Patent”), and U.S. patent application Ser. No. 13/558,108, filed Jul. 25, 2012 (“the '108 Application”), and Ser. No. 13/674,701, filed Nov. 12, 2012 (“the '701 Application”), each of which is incorporated by reference in its entirety.

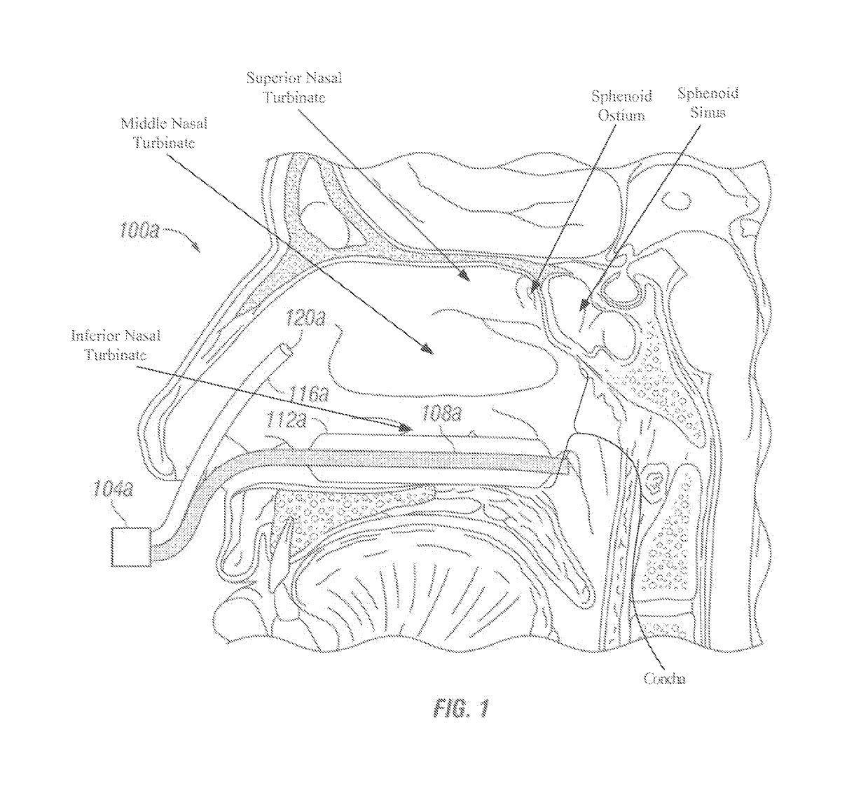

Referring now to FIG. 1, designated by reference numeral 100a is one embodiment of the present cooling devices. In the embodiment shown in FIG. 1, cooling device 100a comprises pump 104a. A pump described in this disclosure or depicted with graphical symbols in the figures can be any pump configured to pump fluid, for example, through one or more tubes coupled to the pump. The term can be used to describe a single pump or a plurality of pumps. The term can also be used to describe a device that introduces fluid into the body of a subject or a device that removes fluid from the body of a subject (e.g., a vacuum). For example, if a plurality of tubes of the present cooling devices are coupled to a pump, each of the plurality of tubes can be coupled to the same or to a different pump. For example, in some embodiments, it may be advantageous to have each of a plurality of tubes coupled to a separate pump to, for example, increase the ability to monitor/adjust the amount of fluid moving through each tube, to monitor/adjust the pressure at any point along each tube, and to monitor/address differences in resistance to flow inside the nasal cavity (e.g., due to the nasal valve) that may affect a desired fluid distribution through the tubes or in the nasal cavity. Further, a pump can be manually operated (e.g., by rotating or reciprocating a pump handle, by depressing the plunger of a syringe, etc.) or non-manually operated (e.g., by actuating an electrically operated pump). Additionally, a pump can be configured to move fluid into a region through a tube or remove fluid from a region through a tube. A pump of the present disclosure can include any pump (and any components of such pump) disclosed in the '787 Patent, the '108 Application, and the '701 Application. Further, the present disclosure includes any components configured to be coupled to and/or integrated with a pump that are disclosed in the '787 Patent, the '108 Application, and the '701 Application, such as tubes, heat exchangers, controllers, and the like.

The embodiment shown in FIG. 1 further comprises blockage 108a configured to be inserted through a nostril of a subject and disposed in the caudal region of the nasal cavity such that resistance to cooling fluid flow in the caudal region of the nasal cavity is increased. A blockage described in this disclosure or depicted in the figures can include any object configured to be disposed in the caudal region of the nasal cavity to increase resistance to cooling fluid flow in the caudal region of the nasal cavity. For example, a blockage can comprise a tube (e.g., a catheter), a stent (e.g., temporary or permanent), an inflatable cuff (e.g., a balloon), a packing material (e.g., gauze, sponges, towels, and the like), and/or a combination thereof. Any blockage of the present disclosure can be configured/sized or configurable/sizable (e.g., by self-expansion, inflation, and the like) to minimize a distance between the blockage and surrounding tissue in the region of the nasal cavity in which the blockage is disposed. For example, a blockage can occupy a majority to substantially all of the empty space in a given region of the nasal cavity. In the embodiment shown, blockage 108a comprises (or is coupled to) inflatable cuff 112a that is positioned such that, after blockage 108a is inserted through a nostril of a subject, inflatable cuff 112a can be inflated to further increase resistance to cooling fluid flow in the caudal region of the nasal cavity (e.g., by applying pressure to at least one of the septum, a middle turbinate, and an inferior turbinate). In some embodiments, a length of an inflatable cuff is substantially equal to a length of a corresponding blockage; and, in other embodiments, a length of an inflatable cuff is less than or greater than a length of a corresponding blockage. In some embodiments, a blockage can be configured to accommodate tissue and/or structures within the nasal cavity (e.g., a turbinate), for example, by comprising an inverse configuration as that tissue and/or structure, as discussed in detail below.

As shown in FIG. 1, blockage 108a is disposed such that it resists fluid flow in the caudal region of the nasal cavity. For example, if fluid flows from the cranial region of the nasal cavity toward the caudal region of the nasal cavity (e.g., due to gravity, a pressure gradient, and the like), blockage 108a can substantially prevent fluid from flowing beyond blockage 108a with respect to the direction of fluid flow. Blockage 108a can be inserted through either nostril of a subject and disposed between the septum and the respective inferior nasal turbinate (as depicted in FIG. 1). In some embodiments, a blockage (e.g., blockage 108a) can be disposed in the inferior nasal meatus and/or the middle nasal meatus. As with other embodiments in this disclosure, blockage 108a can be inserted through either nostril of a subject and disposed between the septum and the respective middle nasal turbinate. In some embodiments, blockage 108a can extend to the choana, whether blockage 108a is disposed between the septum and the middle nasal turbinate or the septum and the inferior nasal turbinate.

The embodiment shown in FIG. 1 also comprises tube 116a configured to be inserted through a nostril of a subject and directed toward the cranial region of the nasal cavity (e.g., toward the superior nasal meatus, the superior nasal turbinate, etc.). Tube 116a can be inserted through either nostril of a subject and directed toward the cranial region of the nasal cavity. In the embodiment shown, tube 116a is coupled to pump 104a such that pump 104a can, if activated, pump fluid through tube 116a and into the nasal cavity. As with any of the tubes in this disclosure, tube 116a can also be coupled to pump 104a such that pump 104a can, if activated, remove fluid, such as from the nasal cavity through tube 116a. Embodiments configured to remove fluid from, for example, the nasal cavity can assist in improving heat exchange (e.g., by removing air that prevents optimal distribution of cooling fluid on mucosal surfaces). Fluid can also be removed by temporary interruption of fluid flow (e.g., which can allow air to rise in a tube) in combination with temporary interruption of any seal (e.g., in the peri-nares area), which allows fluid (e.g., air) to escape into the environment. In the embodiment shown in FIG. 1, tube 116a comprises one opening 120a through which fluid can exit tube 116a; however, in other embodiments, tube 116a can comprise a plurality of openings through which fluid can exit tube 116a (as depicted in other figures). As described in detail in the '787 Patent, the '108 Application, and the '701 Application, fluid used by the cooling devices of the present disclosure can include any free-flowing (e.g., non-nebulized) fluid, such as perfluorocarbons, water, and/or oil mixtures, each of which can further comprise additives, such as simple sugars, organic compounds (e.g., propylene glycol), antibacterial agents, mucosal protectants (e.g., antioxidants, free-radical scavengers, etc.), and/or electrolytes (e.g., potassium, calcium, sodium, and the like). Further, fluid used by the present cooling devices is generally cooled fluid that can range in temperature, for example, from 30° C. to −30° C. In the embodiment shown in FIG. 1, a sufficient volume of fluid can be delivered to the cranial region of the nasal cavity of a subject such that the fluid substantially fills the nasal cavity (e.g., except for any portion occupied or blocked by a blockage (e.g. blockage 108a)). In some embodiments, fluid can passively flow out of the nose and/or mouth of the subject; and in other embodiments, fluid is actively removed from the nose and/or mouth of the subject (e.g., with a suctioning device), as will be discussed further.

In some embodiments (described in detail with respect to other embodiments), blockage 108a and tube 116a are each coupled to an inflatable cuff such that, after blockage 108a and tube 116a are each inserted through a nostril of a subject, the inflatable cuff can be inflated to further increase resistance to cooling fluid flow in the caudal region of the nasal cavity. For example, the inflatable cuff can extend between blockage 108a and tube 116a and can, for example, apply pressure to at least one of the septum, a middle turbinate, an inferior turbinate, and other tissue or structures in the nasal cavity near blockage 108a and/or tube 116a. Further, device 100a, blockage 108a, and tube 116a can comprise and/or be coupled to any of the features and components described with respect to the figures and embodiments of this disclosure.

In the embodiment shown in FIG. 1, device 100a is configured to pump fluid into the nasal cavity of the subject to cool the brain of the subject. In some embodiments, device 100a is configured to cool the brain of the subject until a brain to body temperature gradient of at least 1° C. is achieved (e.g., a temperature gradient of 1° C., 2° C., 3° C., 4° C., 5° C., 6° C., 7° C., 8° C., 9° C., 10° C., or more). In some embodiments, device 100a is configured to cool the brain of the subject with fluid that is cooler than room temperature (e.g., cooler than 28° C., 26° C., 24° C., 22° C., 20° C., 18° C., or cooler). In some embodiments, device 100a is configured to cool the brain of the subject for at least one hour (e.g., 1 hour, 2 hours, 3 hours, 4 hours, 5 hours, 6 hours, 7 hours, 8 hours, 9 hours, 10 hour, or more). In some embodiments, device 100a is configured to cool the brain of the subject with at least 250 milliliters of fluid (e.g., 250 milliliters, 500 milliliters, 750 milliliters, 1 liter, 1.25 liters, 1.5 liters, 1.75 liters, 2 liters, or more).

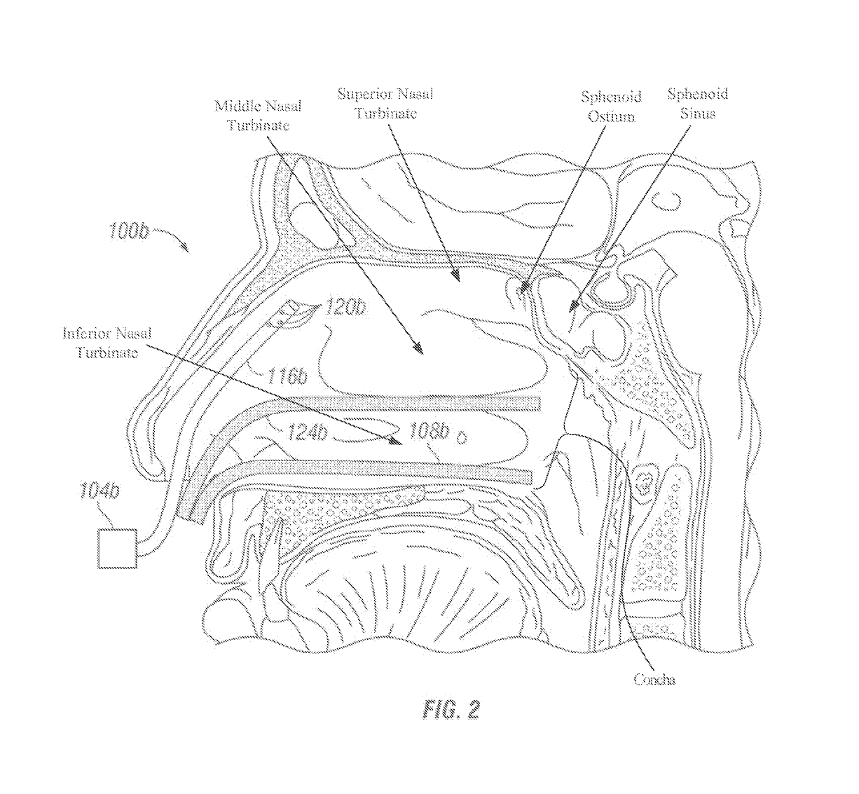

Referring now to FIG. 2, designated by reference numeral 100b is another embodiment of the present cooling devices. Similar to FIG. 1, device 100b comprises pump 104b, blockage 108b configured to be inserted through a nostril of a subject and disposed in the caudal region of the nasal cavity such that resistance to cooling fluid flow in the caudal region of the nasal cavity is increased, and tube 116b configured to be inserted through a nostril of a subject and directed toward the cranial region of the nasal cavity (e.g., toward the superior nasal meatus, the superior nasal turbinate, etc.). In the embodiment shown in FIG. 2, tube 116b is coupled to pump 104b such that pump 104b can, if activated, pump fluid through tube 116b and into the nasal cavity. In the embodiment shown in FIG. 2, tube 116b comprises plurality of openings 120b through which fluid can exit tube 116b. In other embodiments, tube 116b can comprise one opening (e.g., as shown in FIG. 1 with tube 116a) through which fluid can exit tube 116b.

The embodiment shown in FIG. 2 further comprises blockage 124b. Blockage 108b and 124b are each disposed such that the blockages resist fluid flow in the caudal region of the nasal cavity. For example, if fluid flows from the cranial region of the nasal cavity toward the caudal region of the nasal cavity (e.g., due to gravity, a pressure gradient, and the like), blockage 108b and blockage 124b can substantially prevent fluid from flowing beyond each blockage with respect to the direction of fluid flow. In the embodiment shown, blockage 108b is disposed between the septum and the inferior nasal turbinate, and blockage 124b is disposed between the septum and the middle nasal turbinate. However, in other embodiments, blockage 108b and blockage 124b can both be disposed between the septum and the inferior nasal turbinate or between the septum and the middle nasal turbinate (e.g., in a stacked configuration). In still other embodiments, blockage 108b can be disposed between the inferior nasal turbinate and the septum and blockage 124b can be disposed in the inferior nasal meatus. And in other embodiments, blockage 108b can be disposed between the middle nasal turbinate and the septum and blockage 124b can be disposed in the middle nasal meatus. Other variations can similarly be employed (e.g., blockage 108b disposed between the inferior nasal turbinate and the septum and blockage 124b disposed in the middle nasal meatus). In some embodiments, blockage 108b and blockage 124b extend to the choana, whether blockage 108b or blockage 124b is disposed between the septum and the middle nasal turbinate or the septum and the inferior nasal turbinate. Blockage 108b, blockage 124b, and tube 116b can each be inserted through either nostril of a subject and in any of the described configurations. Further, device 100b, blockage 108b, blockage 124b, and tube 116b can comprise and/or be coupled to any of the features and components described with respect to the figures and embodiments of this disclosure.

As with FIG. 1, blockage 108b and/or blockage 124b can comprise (or be coupled to) an inflatable cuff that is positioned such that, after blockage 108b and/or blockage 124b is inserted through a nostril of a subject, the inflatable cuff can be inflated to further increase resistance to cooling fluid flow in the caudal region of the nasal cavity (e.g., by applying pressure to at least one of the septum, a middle turbinate, and an inferior turbinate). In some embodiments, blockage 108b and tube 116b are each coupled to an inflatable cuff such that, after blockage 108b and tube 116b are each inserted through a nostril of a subject, the inflatable cuff can be inflated to further increase resistance to cooling fluid flow in the caudal region of the nasal cavity. Similarly, blockage 124b and another tube (e.g., a tube similar to tube 116b) can be coupled to an inflatable cuff such that, after blockage 124b and the other tube are each inserted through a nostril of a subject, the inflatable cuff can be inflated to further increase resistance to cooling fluid flow in the caudal region of the nasal cavity. For example, an inflatable cuff can extend between blockage 108b and tube 116b and an inflatable cuff can extend between blockage 124b and the other tube such that, when inflated, pressure can be applied to at least one of the septum, a middle turbinate, an inferior turbinate, and other tissue and/or structures.

In the embodiment shown in FIG. 2, device 100b is configured to pump fluid into the nasal cavity of the subject to cool the brain of the subject. In some embodiments, device 100b is configured to cool the brain of the subject until a brain to body temperature gradient of at least 1° C. is achieved (e.g., a temperature gradient of 1° C., 2° C., 3° C., 4° C., 5° C., 6° C., 7° C., 8° C., 9° C., 10° C., or more). In some embodiments, device 100b is configured to cool the brain of the subject with fluid that is cooler than room temperature (e.g., cooler than 28° C., 26° C., 24° C., 22° C., 20° C., 18° C., or cooler). In some embodiments, device 100b is configured to cool the brain of the subject for at least one hour (e.g., 1 hour, 2 hours, 3 hours, 4 hours, 5 hours, 6 hours, 7 hours, 8 hours, 9 hours, 10 hour, or more). In some embodiments, device 100b is configured to cool the brain of the subject with at least 250 milliliters of fluid (e.g., 250 milliliters, 500 milliliters, 750 milliliters, 1 liter, 1.25 liters, 1.5 liters, 1.75 liters, 2 liters, or more).

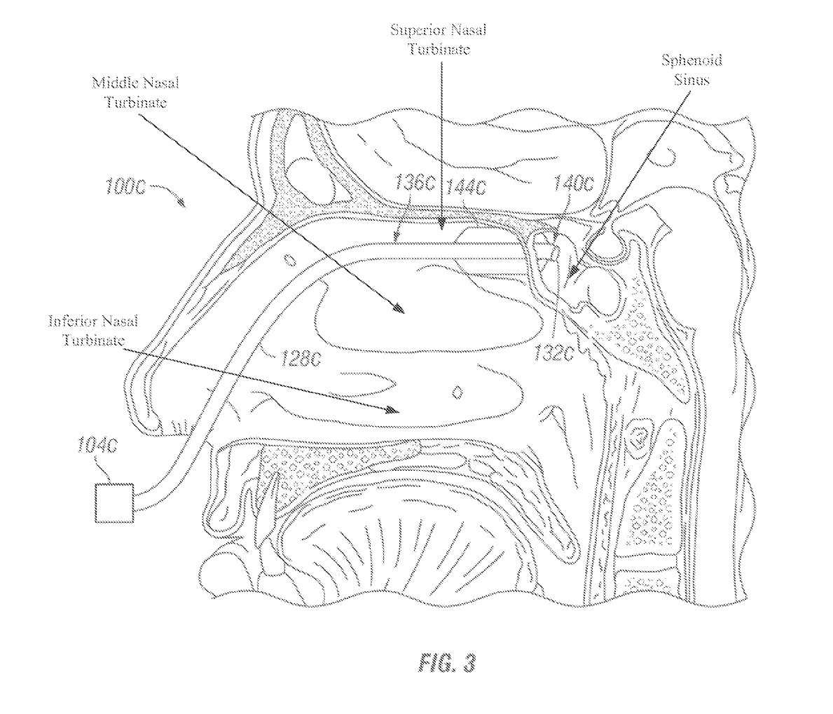

Referring now to FIG. 3, designated by reference numeral 100c is another embodiment of the present cooling devices. Cooling device 100c comprises pump 104c and sphenoid sinus tube 128c. Sphenoid sinus tube 128c is coupled to pump 104c and configured to be inserted through either nostril of a subject and into a respective sphenoid sinus of the subject such that pump 104c can, if activated, pump fluid through sphenoid sinus tube 128c and into the respective sphenoid sinus. In the embodiment shown in FIG. 3, sphenoid sinus tube 128c comprises one opening 132c through which fluid can exit sphenoid sinus tube 128c; however, in other embodiments, sphenoid sinus tube 128c can comprise a plurality of openings through which fluid can exit sphenoid sinus tube 128c (as depicted in other figures).

A sufficient volume of fluid can be delivered to a sphenoid sinus of a subject such that fluid substantially fills the sphenoid sinus. In some embodiments, fluid can passively flow out of the sphenoid ostium of the sphenoid sinus in which sphenoid sinus tube 128c is inserted. In some embodiments, where the sphenoid sinus associated with a first nostril is in fluid communication with the sphenoid sinus associated with a second nostril, fluid can passively flow out of either sphenoid ostium. In some embodiments, cooling device 100c can comprise a second sphenoid sinus tube configured to be inserted through a nostril of a subject and into a sphenoid sinus of the subject. In such an embodiment, device 100c can be configured to remove (e.g., with a suctioning device) fluid from the sphenoid sinus through the second sphenoid sinus tube. In some embodiments, where the sphenoid sinus associated with a first nostril is in fluid communication with the sphenoid sinus associated with a second nostril, fluid can be actively removed from either sphenoid sinus through the second tube. In another embodiment, sphenoid sinus tube 128c can comprise a multi-lumen catheter such that pump 104c can, if activated, pump fluid through a first lumen of sphenoid sinus tube 128c and into the sphenoid sinus, and fluid can be removed from the sphenoid sinus through a second lumen of sphenoid sinus tube 128c.

Any tube in this application can comprise any suitable, biocompatible material, whether the tube is directed toward the cranial region of the nasal cavity or inserted into the sphenoid sinus. For example, in some embodiments, sphenoid sinus tube 128c comprises a substantially malleable material. In some embodiments, sphenoid sinus tube 128c comprises a substantially rigid material. In other embodiments, sphenoid sinus tube 128c comprises first portion 136c, which comprises a substantially rigid material and second portion 140c, which comprises a substantially malleable material. In some embodiments, sphenoid sinus tube 128c can comprise a friction-reducing coating, such as Teflon, to enable sphenoid sinus tube 128c to be more easily inserted through a sphenoid ostium.

Sphenoid sinus tube 128c can comprise or be coupled to various other components. For example, sphenoid sinus tube 128c can be coupled to a pressure sensor that is configured to detect if pressure in the sphenoid sinus meets or exceeds a threshold pressure (e.g., to prevent damage to tissue and/or structures defining the sphenoid sinus). If pressure in the sphenoid sinus meets or exceeds a threshold pressure, the cooling device (e.g., cooling device 100c) can, for example, reduce the flow rate of fluid through sphenoid sinus tube 128c (e.g., automatically or by user input). Sphenoid sinus tube 128c can also comprise or be coupled to a stopper configured to limit the extent to which sphenoid sinus tube 128c can enter the sphenoid sinus (e.g., to prevent damage to tissue and/or structures defining the sphenoid sinus).

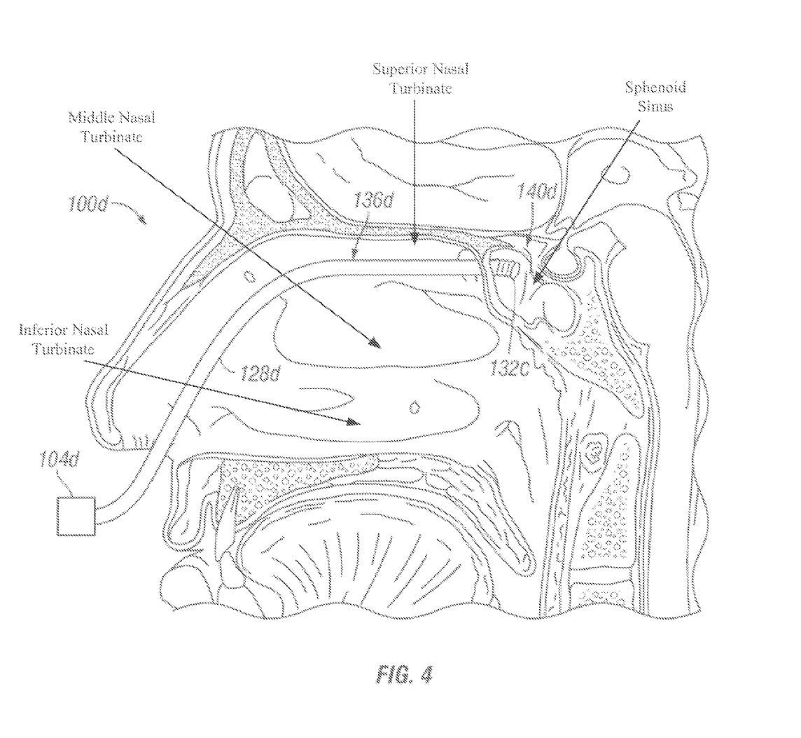

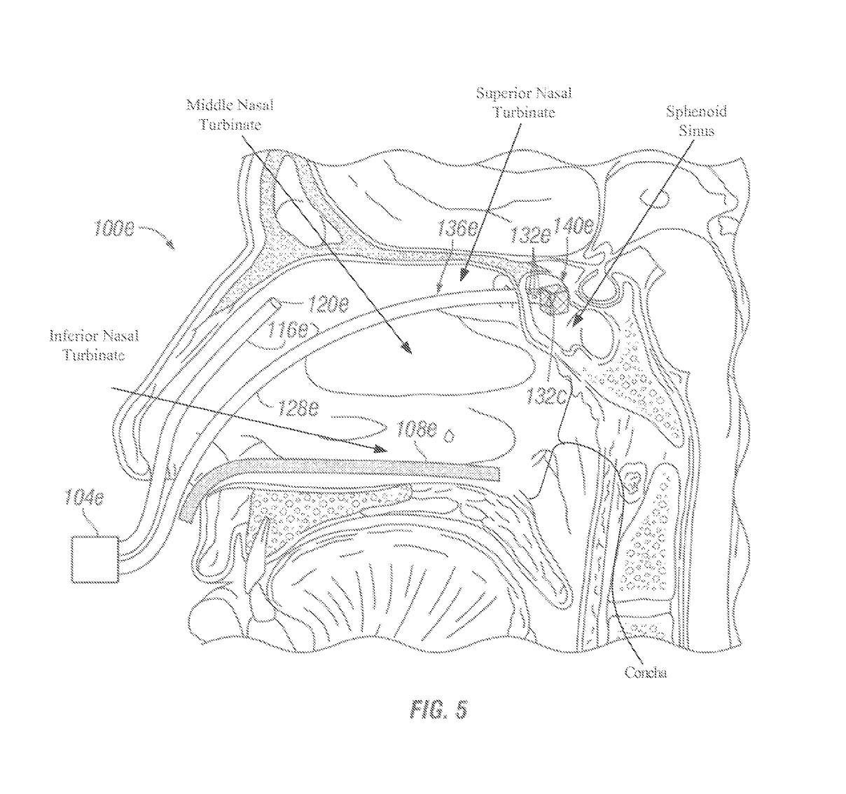

Sphenoid sinus tube 128c can also be configured such that, after sphenoid sinus tube 128c is inserted into the sphenoid sinus of the subject, a portion of sphenoid sinus tube 128c prevents sphenoid sinus tube 128c from exiting the sphenoid sinus. For example, second portion 140c of sphenoid sinus tube 128c can be configured to prevent sphenoid sinus tube 128c from exiting the sphenoid sinus. As depicted in FIG. 4, after sphenoid sinus tube 128c is inserted into the sphenoid sinus of the subject, second portion 140d of sphenoid sinus tube 128d coils (e.g., into a pig-tail configuration) to prevent sphenoid sinus 128d tube from exiting the sphenoid sinus of the subject. For example, second portion 140d of sphenoid sinus tube 128d can be biased toward such a coiled/pig-tail configuration, and a stylet over which sphenoid sinus tube 128d is disposed when introducing sphenoid sinus tube 128d to the sphenoid sinus can prevent second portion 140d of sphenoid sinus tube 128d from coiling. After sphenoid sinus tube 128d is introduced to the sphenoid sinus and the stylet is removed, second portion 140d of sphenoid sinus tube 128d is permitted to coil. In other embodiments, after sphenoid sinus tube 128c is inserted into the sphenoid sinus of the subject, a portion of the sphenoid sinus tube can expand (e.g., into a basket-like configuration, as depicted in FIG. 5) to prevent the sphenoid sinus tube from exiting the sphenoid sinus of the subject. In some embodiments, after sphenoid sinus tube 128c is inserted into the sphenoid sinus of the subject, sphenoid sinus tube 128c is coupled to the nose (e.g., clipped to a nostril) of the subject such that sphenoid sinus tube 128c is prevented from exiting the sphenoid sinus of the subject (e.g., and substantially prevented from exiting the nostril).

As depicted in FIG. 3, sphenoid sinus tube 128c also can comprise (or be coupled to) inflatable cuff 144c. Inflatable cuff 144c is positioned such that, after sphenoid sinus tube 128c is inserted into the sphenoid sinus of the subject, inflatable cuff 144c can be inflated (in some instances, temporarily, for example, prior to fluid entering the sphenoid sinus) to engage tissue defining a sphenoid ostium and, therefore, increasing the area of that sphenoid ostium. In some embodiments, inflatable cuff 144c is inflated to increase the area of a sphenoid ostium, and then deflated prior to introducing fluid through sphenoid sinus tube 128c into the sphenoid sinus (e.g., to permit fluid to passively flow out of the sphenoid sinus). In other embodiments, a sphenoid ostium can be blocked or partially blocked (e.g., by inflating or partially inflating inflatable cuff 144c or by some other blockage) while fluid is introduced through sphenoid sinus tube 128c into the sphenoid sinus (e.g., to increase a volume of fluid in the sphenoid sinus and, thus, increase the surface area of the sphenoid sinus in contact with the fluid). Further, devices 100c and 100d and sphenoid sinus tubes 128c and 128d can comprise and/or be coupled to any of the features and components described with respect to the figures and embodiments of this disclosure.

In the embodiments shown in FIGS. 3-4, devices 100c and 100d are configured to pump fluid into the nasal cavity of the subject to cool the brain of the subject. In some embodiments, devices 100c and 100d are configured to cool the brain of the subject until a brain to body temperature gradient of at least 1° C. is achieved (e.g., a temperature gradient of 1° C., 2° C., 3° C., 4° C., 5° C., 6° C., 7° C., 8° C., 9° C., 10° C., or more). In some embodiments, devices 100c and 100d are configured to cool the brain of the subject with fluid that is cooler than room temperature (e.g., cooler than 28° C., 26° C., 24° C., 22° C., 20° C., 18° C., or cooler). In some embodiments, devices 100c and 100d are configured to cool the brain of the subject for at least one hour (e.g., 1 hour, 2 hours, 3 hours, 4 hours, 5 hours, 6 hours, 7 hours, 8 hours, 9 hours, 10 hour, or more). In some embodiments, devices 100c and 100d are configured to cool the brain of the subject with at least 250 milliliters of fluid (e.g., 250 milliliters, 500 milliliters, 750 milliliters, 1 liter, 1.25 liters, 1.5 liters, 1.75 liters, 2 liters, or more).

Referring now to FIG. 5, designated by reference numeral 100e is another embodiment of the present cooling devices. Similar to FIG. 1, device 100e comprises pump 104e, blockage 108e configured to be inserted through a nostril of a subject and disposed in the caudal region of the nasal cavity such that resistance to cooling fluid flow in the caudal region of the nasal cavity is increased, and tube 116e configured to be inserted through a nostril of a subject and directed toward the cranial region of the nasal cavity (e.g., toward the superior nasal meatus, the superior nasal turbinate, etc.). In the embodiment shown in FIG. 5, tube 116e is coupled to pump 104e such that pump 104e can, if activated, pump fluid through tube 116e and into the nasal cavity. In the embodiment shown in FIG. 5, tube 116e comprises one opening 120e through which fluid can exit tube 116e; however, as in other embodiments, tube 116e can comprise a plurality of openings (e.g., as shown in FIG. 2 with tube 116b) through which fluid can exit tube 116e. The embodiment shown in FIG. 5 further comprises sphenoid sinus tube 128e coupled to pump 104e and configured to be inserted through a nostril of a subject and into a respective sphenoid sinus of the subject such that pump 104e can, if activated, pump fluid through sphenoid sinus tube 128e and into the respective sphenoid sinus. In the embodiment shown in FIG. 5, sphenoid sinus tube 128e comprises plurality of openings 132e through which fluid can exit sphenoid sinus tube 128e; however, in other embodiments, sphenoid sinus tube 128e can comprise one opening through which fluid can exit sphenoid sinus tube 128e (as with sphenoid sinus tube 128c in FIG. 3). In some embodiments, sphenoid sinus tube 128e can be coupled to pump 104e (e.g., a roller pump, a wall suction device, and the like) and configured to be inserted through a nostril of a subject and into a respective sphenoid sinus of the subject such that pump 104e can, if activated, pump fluid out of the sphenoid sinus (e.g., creating negative pressure inside the sphenoid sinus) through sphenoid sinus tube 128e. As described in detail above, the depiction of pump 104e in FIG. 5, as well as any other depiction of pumps in the figures, can represent more than one pump, more than one type of pump, or one pump configured to perform more than one operation. Therefore, in the present embodiment, sphenoid sinus tube 128e can be coupled to pump 104e, which is configured to remove fluid from the sphenoid sinus, and tube 116e can be coupled to pump 104e, which is configured to pump fluid into the nasal cavity. In such an embodiment, fluid in the nasal cavity can move into the sphenoid sinus through the sphenoid ostium, for example, due to a pressure gradient. In the embodiment shown in FIG. 5, sphenoid sinus tube 128e comprises plurality of openings 132e through which fluid can enter sphenoid sinus tube 128e; however, in other embodiments, sphenoid sinus tube 128e can comprise one opening through which fluid can enter sphenoid sinus tube 128e (as with sphenoid sinus tube 128c in FIG. 3). Such an embodiment in which fluid enters the sphenoid sinus, for example, due to a pressure gradient fluid, and is removed from the sphenoid sinus via a sphenoid sinus tube can be implemented in any of the other embodiments and configurations described throughout this disclosure. As in FIGS. 1-4, blockage 108e, tube 116e, and sphenoid sinus tube 128e can each be inserted through either nostril of a subject and in any of the described configurations, and none are required to be in the same nostril at the same time. Further, device 100e, blockage 108e, tube 116e, and sphenoid sinus tube 128e can comprise and/or be coupled to any of the features and components described with respect to the figures and embodiments of this disclosure.

In the embodiment shown in FIG. 5, sphenoid sinus tube 128e is configured such that, after sphenoid sinus tube 128e is inserted into the sphenoid sinus of the subject, a portion of sphenoid sinus tube 128e prevents sphenoid sinus tube 128e from exiting the sphenoid sinus. For example, second portion 140e of sphenoid sinus tube 128e can be configured to prevent sphenoid sinus tube 128e from exiting the sphenoid sinus. As depicted in FIG. 5, after sphenoid sinus tube 128e is inserted into the sphenoid sinus of the subject, second portion 140e of sphenoid sinus tube 128e expands (e.g., into a basket configuration) to prevent sphenoid sinus tube 128e from exiting the sphenoid sinus of the subject.

In the embodiment shown in FIG. 5, device 100e is configured to pump fluid into the nasal cavity of the subject to cool the brain of the subject. In some embodiments, device 100e is configured to cool the brain of the subject until a brain to body temperature gradient of at least 1° C. is achieved (e.g., a temperature gradient of 1° C., 2° C., 3° C., 4° C., 5° C., 6° C., 7° C., 8° C., 9° C., 10° C., or more). In some embodiments, device 100e is configured to cool the brain of the subject with fluid that is cooler than room temperature (e.g., cooler than 28° C., 26° C., 24° C., 22° C., 20° C., 18° C., or cooler). In some embodiments, device 100e is configured to cool the brain of the subject for at least one hour (e.g., 1 hour, 2 hours, 3 hours, 4 hours, 5 hours, 6 hours, 7 hours, 8 hours, 9 hours, 10 hour, or more). In some embodiments, device 100e is configured to cool the brain of the subject with at least 250 milliliters of fluid (e.g., 250 milliliters, 500 milliliters, 750 milliliters, 1 liter, 1.25 liters, 1.5 liters, 1.75 liters, 2 liters, or more).

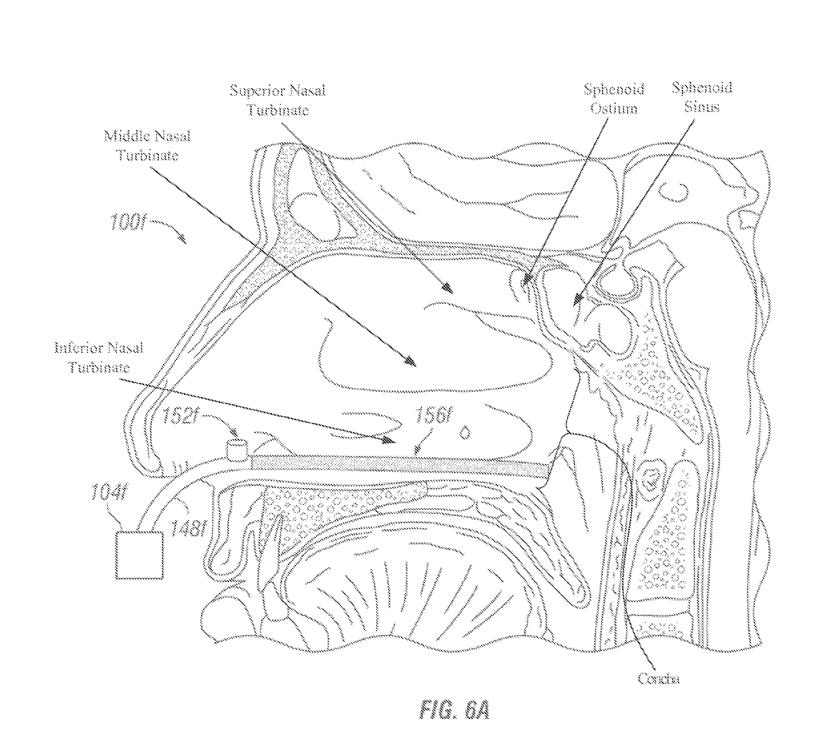

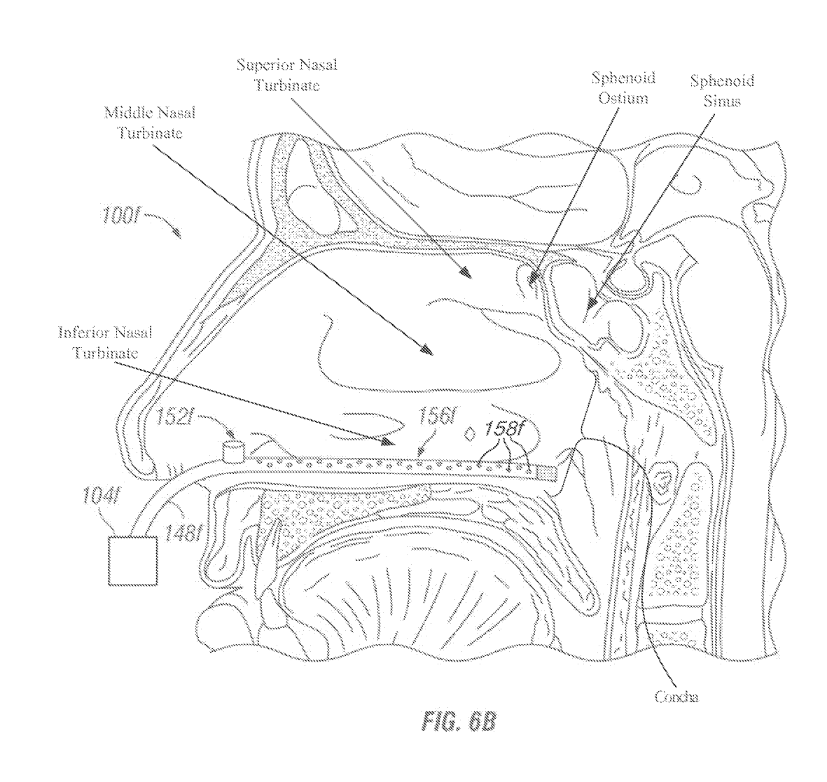

Referring now to FIG. 6A, designated by reference numeral 100f is another embodiment of the present cooling devices. In the embodiment shown, device 100f comprises pump 104f coupled to nasal insert 148f such that pump 104f can, if activated, pump fluid through nasal insert 148f. In the embodiment shown, nasal insert 148f comprises first portion 152f configured to be inserted through a nostril of a subject and directed toward the cranial region of the nasal cavity. Nasal insert 148f further comprises second portion 156f configured to be inserted through a nostril of a subject and disposed in the caudal region of the nasal cavity such that resistance to cooling fluid flow in the caudal region of the nasal cavity is increased. In some embodiments (e.g., FIG. 6A), nasal insert 148f is configured to prevent fluid from flowing through second portion 156f of nasal insert 148f; and in other embodiments (e.g., FIG. 6B), nasal insert 148f is configured to permit fluid to flow through part or all of second portion 156f of nasal insert 148f. For example, in the embodiment shown in FIG. 6B, second portion 156f of nasal insert 148f comprises plurality of openings 158f. In such an embodiment, second portion 156f of nasal insert 148f can be positioned such that, if fluid moves through second portion 156f of the nasal insert 148f, fluid can exit second portion 156f of nasal insert 148f through plurality of openings 158f and toward the cranial region of the nasal cavity. In the embodiment shown, fluid is prevented from exiting at the distal end of second portion 156f of nasal insert 148f; however, in other embodiments, the distal end of second portion 156f of nasal insert 148f can be configured to permit fluid to exit. As depicted in FIGS. 6A and 6B, second portion 156f of nasal insert 148f is configured to be disposed between the septum and an inferior nasal turbinate of the subject. In other embodiments, second portion 156f of nasal insert 148f is configured to be disposed between the septum and a middle nasal turbinate of the subject. As depicted with blockage 108a in FIG. 1, nasal insert 148f can extend to the choana, whether nasal insert 148f is disposed between the septum and the middle nasal turbinate or the septum and the inferior nasal turbinate.

In some embodiments, device 100f can comprise plurality of nasal inserts 148f, where each of the plurality of nasal inserts 148f comprises first portion 152f configured to be inserted through a nostril of a subject and directed toward the cranial region of the nasal cavity, and each of the plurality of nasal inserts 148f comprises second portion 156f configured to be inserted through a nostril of a subject and disposed in the caudal region of the nasal cavity such that resistance to cooling fluid flow in the caudal region of the nasal cavity is increased. Second portion 156f of each of plurality of nasal inserts 148f can be configured to be disposed between the septum and an inferior nasal turbinate and/or the septum and a middle nasal turbinate. For example, in one embodiment, device 100f can comprise one nasal insert 148f in a first nostril between the septum and the respective inferior nasal turbinate, one nasal insert 148f in a first nostril between the septum and the respective middle nasal turbinate, one nasal insert 148f in a second nostril between the septum and the respective inferior nasal turbinate, and one nasal insert 148f in a second nostril between the septum and the respective middle nasal turbinate. Any number of similar configurations can be achieved with device 100f. In some embodiments, similarly to those described below, second portion 156f of nasal inserts 148f can comprise a substantially semi-cylindrical configuration that is substantially inverse to the shape of a turbinate with which each blockage is in contact. For example, the substantially semi-cylindrical configuration of permits a portion of second portion 156f to be disposed between the septum and the inferior nasal turbinate and a portion of each blockage to extend into the inferior nasal meatus (e.g., or a portion to be disposed between the septum and the middle nasal turbinate and a portion to extend into the middle nasal meatus). In other embodiments, as will be described below with respect to other tubes of the present devices, a nasal insert can also comprise a circle configuration, an oval configuration, a teardrop configuration, and a frustum configuration, among other configurations. Device 100f and/or nasal insert 148f can comprise and/or be coupled to any of the features and components described with respect to the figures and embodiments of this disclosure.

In the embodiment shown in FIGS. 6A and 6B, device 100f is configured to pump fluid into the nasal cavity of the subject to cool the brain of the subject. In some embodiments, device 100f is configured to cool the brain of the subject until a brain to body temperature gradient of at least 1° C. is achieved (e.g., a temperature gradient of 1° C., 2° C., 3° C., 4° C., 5° C., 6° C., 7° C., 8° C., 9° C., 10° C., or more). In some embodiments, device 100f is configured to cool the brain of the subject with fluid that is cooler than room temperature (e.g., cooler than 28° C., 26° C., 24° C., 22° C., 20° C., 18° C., or cooler). In some embodiments, device 100f is configured to cool the brain of the subject for at least one hour (e.g., 1 hour, 2 hours, 3 hours, 4 hours, 5 hours, 6 hours, 7 hours, 8 hours, 9 hours, 10 hour, or more). In some embodiments, device 100f is configured to cool the brain of the subject with at least 250 milliliters of fluid (e.g., 250 milliliters, 500 milliliters, 750 milliliters, 1 liter, 1.25 liters, 1.5 liters, 1.75 liters, 2 liters, or more).

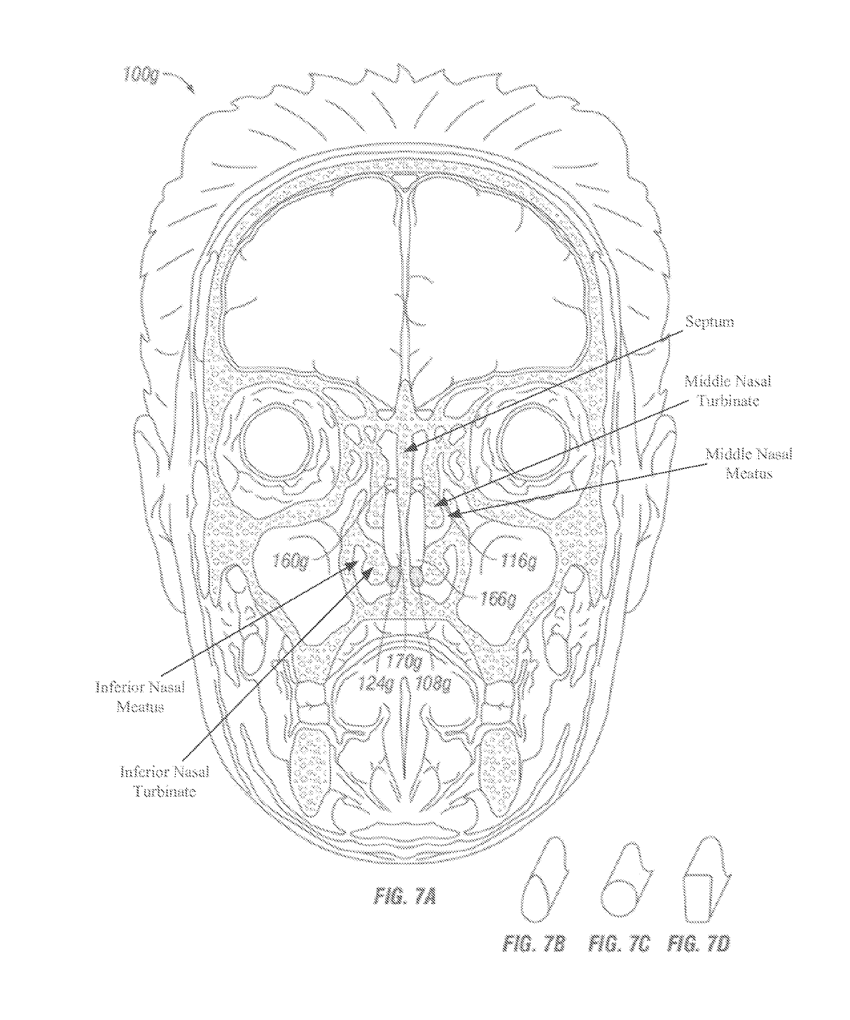

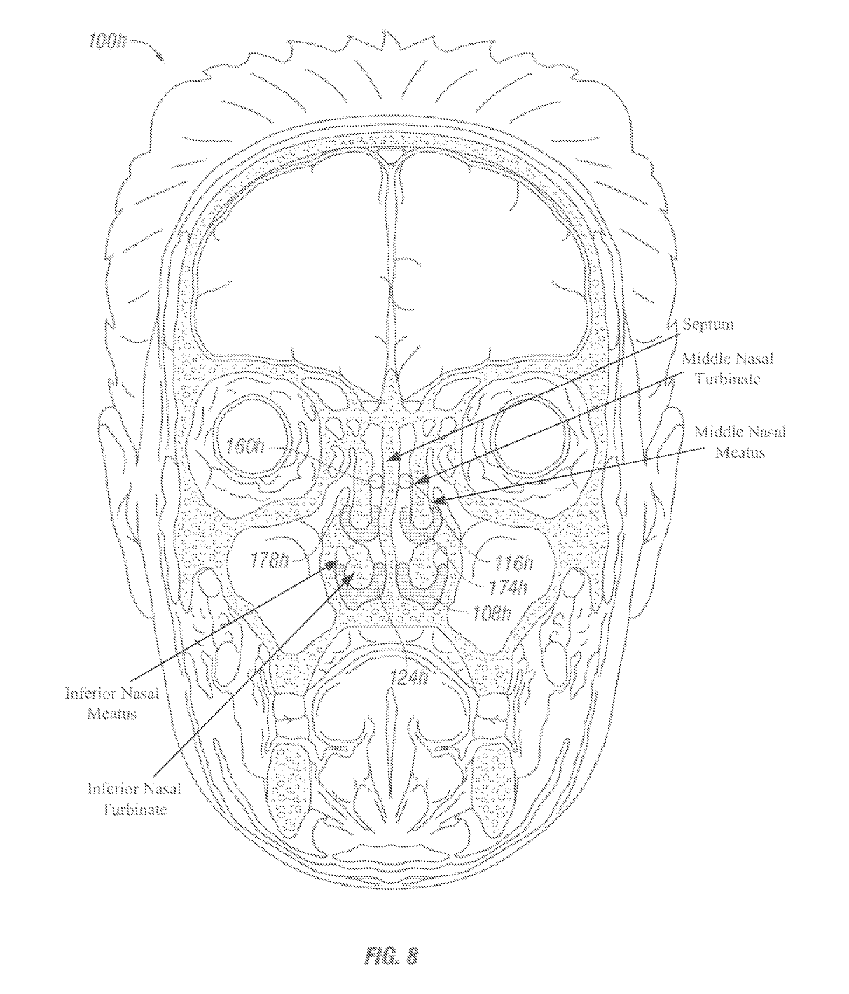

Referring now to FIG. 7A, designated by reference numeral 100g is another embodiment of the present cooling devices. Similar to FIG. 2, device 100g comprises pump 104g, blockage 108g configured to be inserted through a nostril of a subject and disposed in the caudal region of the nasal cavity such that resistance to cooling fluid flow in the caudal region of the nasal cavity is increased, and tube 116g configured to be inserted through a nostril of a subject and directed toward the cranial region of the nasal cavity (e.g., toward the superior nasal meatus, the superior nasal turbinate, etc.). Device 100g further comprises blockage 124g, which is also disposed such that blockage 124g resists fluid flow to the caudal region of the nasal cavity. For example, if fluid flows from the cranial region of the nasal cavity toward the caudal region of the nasal cavity (e.g., due to gravity, a pressure gradient, and the like), blockage 108g and blockage 124g can substantially prevent fluid from flowing beyond each blockage with respect to the direction of fluid flow. In the embodiment shown, blockage 108g and blockage 124g are each disposed between the septum and the inferior nasal turbinate of different nostrils. However, in other embodiments, blockage 108g is disposed between the septum and the inferior nasal turbinate of a first nostril, and blockage 124g is disposed between the septum and the middle nasal turbinate of a second nostril. In still other embodiments, blockage 108g and blockage 124g can be disposed in a meatus of either nostril (e.g., an inferior nasal meatus, a middle nasal meatus, etc.). In some embodiments, blockage 108g and blockage 124g extend to the choana, whether blockage 108g or blockage 124g is disposed between the septum and the middle nasal turbinate or the septum and the inferior nasal turbinate.

In the embodiment shown in FIG. 7A, device 100g further comprises tube 160g configured to be inserted through a nostril of a subject and directed toward the cranial region of the nasal cavity (e.g., toward the superior nasal meatus, the superior nasal turbinate, etc.). Tube 116g and tube 160g are each coupled to pump 104g such that pump 104g can, if activated, pump fluid through tube 116g and tube 160g and into the nasal cavity. As with other embodiments, tube 116g and tube 160g can each comprise one opening or a plurality of openings through which fluid can exit the tubes. Blockage 108g, tube 116g, blockage 124g, and tube 160g can each be inserted through either nostril of a subject and in any of the described configurations, and none are required to be in the same nostril at the same time. Further, device 100g, blockage 108g, tube 116g, blockage 124g, and tube 160g can comprise and/or be coupled to any of the features and components described with respect to the figures and embodiments of this disclosure.

As above, blockage 108g and/or blockage 124g can comprise (or be coupled to) an inflatable cuff that is positioned such that, after blockage 108g and/or blockage 124g is inserted through a nostril of a subject, the inflatable cuff can be inflated to further increase resistance to cooling fluid flow in the caudal region of the nasal cavity (e.g., by applying pressure to at least one of the septum, a middle turbinate, and an inferior turbinate). In the embodiment shown, blockage 108g and tube 116g are each coupled to inflatable cuff 166g such that, after blockage 108g and tube 116g are each inserted through a nostril of a subject, the inflatable cuff can be inflated to further increase resistance to cooling fluid flow in the caudal region of the nasal cavity. Similarly, blockage 124g and tube 160g are coupled to inflatable cuff 170g such that, after blockage 124g and tube 160g are each inserted through a nostril of a subject, inflatable cuff 170g can be inflated to further increase resistance to cooling fluid flow in the caudal region of the nasal cavity. For example, inflatable cuff 166g extends between blockage 108g and tube 116g, and inflatable cuff 170g extends between blockage 124g and tube 160g, such that, when inflated, pressure can be applied to at least one of the septum, a middle turbinate, an inferior turbinate, and other tissue and/or structures.

The blockages, tubes, and nasal inserts of the present cooling devices can comprise a number of configurations. For example, as depicted in FIGS. 7A and 7C, tubes 116g and 160g comprise a circle configuration. Also in FIG. 7A, blockages 108g and 124g comprise an oval configuration. In some instances, a blockage, tube, and/or nasal insert can intrinsically comprise an oval configuration; and in other embodiments, a blockage, tube, and/or nasal insert may appear to comprise an oval configuration due to pressure applied by surrounding body structures and tissues (e.g., a blockage, tube, and/or nasal insert may be “squished” into an oval configuration). As depicted in FIG. 7B, a blockage, tube, and/or nasal insert also can comprise a teardrop configuration. As shown in FIG. 7D, a blockage, tube, and/or nasal insert further can comprise a frustum configuration.

In the embodiment shown in FIG. 7A, device 100g is configured to pump fluid into the nasal cavity of the subject to cool the brain of the subject. In some embodiments, device 100g is configured to cool the brain of the subject until a brain to body temperature gradient of at least 1° C. is achieved (e.g., a temperature gradient of 1° C., 2° C., 3° C., 4° C., 5° C., 6° C., 7° C., 8° C., 9° C., 10° C., or more). In some embodiments, device 100g is configured to cool the brain of the subject with fluid that is cooler than room temperature (e.g., cooler than 28° C., 26° C., 24° C., 22° C., 20° C., 18° C., or cooler). In some embodiments, device 100g is configured to cool the brain of the subject for at least one hour (e.g., 1 hour, 2 hours, 3 hours, 4 hours, 5 hours, 6 hours, 7 hours, 8 hours, 9 hours, 10 hour, or more). In some embodiments, device 100g is configured to cool the brain of the subject with at least 250 milliliters of fluid (e.g., 250 milliliters, 500 milliliters, 750 milliliters, 1 liter, 1.25 liters, 1.5 liters, 1.75 liters, 2 liters, or more).

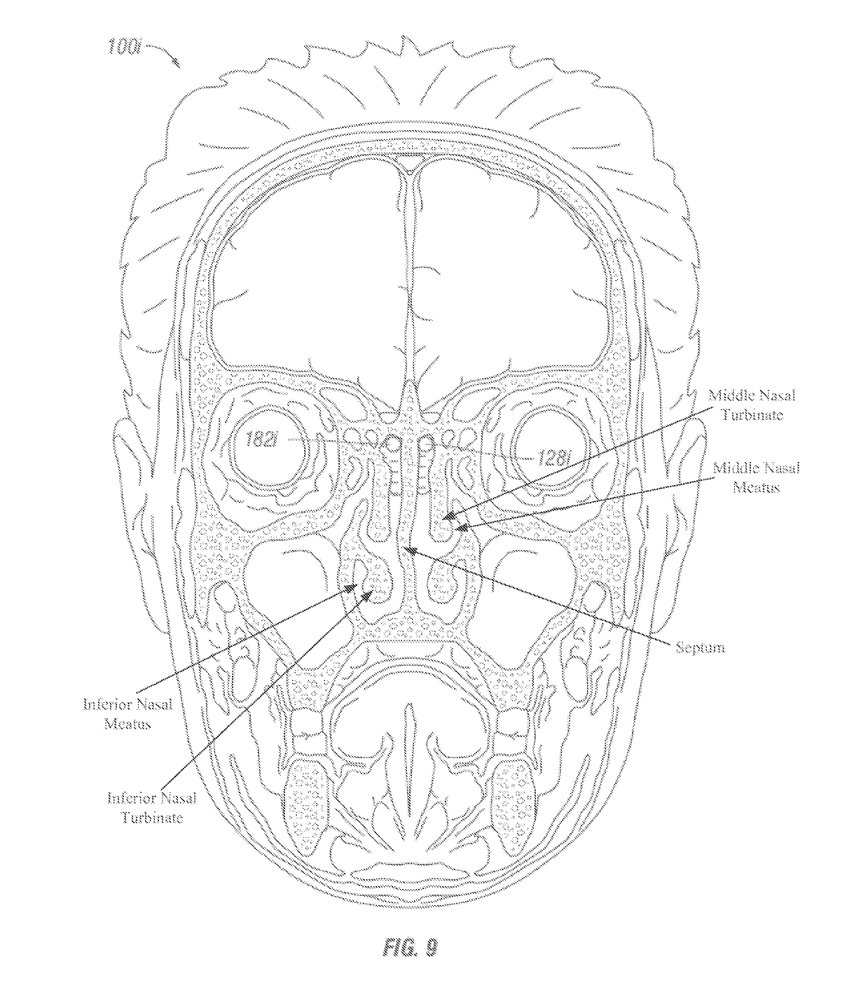

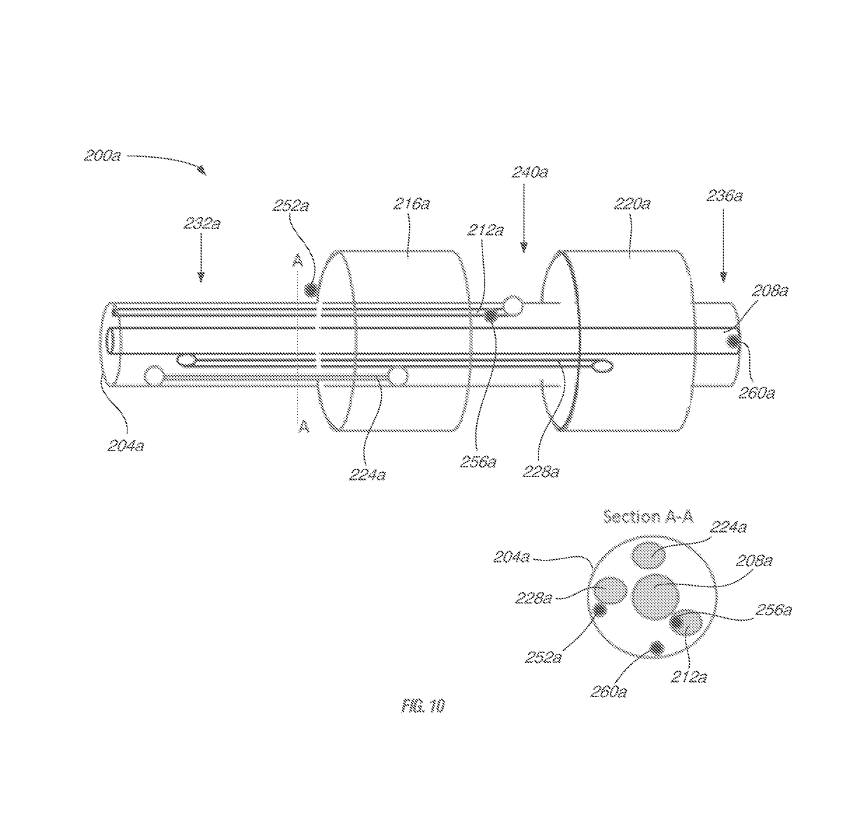

Referring now to FIG. 8, designated by reference numeral 100h is another embodiment of the present cooling devices. Similar to FIG. 7A, device 100h comprises pump 104h, blockages 108h and 124h configured to be inserted through a nostril of a subject and disposed in the caudal region of the nasal cavity such that resistance to cooling fluid flow in the caudal region of the nasal cavity is increased, and tubes 116h and 160h configured to be inserted through a nostril of a subject and directed toward the cranial region of the nasal cavity (e.g., toward the superior nasal meatus, the superior nasal turbinate, etc.). Device 100h further comprises blockage 174h and blockage 178h, each disposed such that the blockage resists fluid flow to the caudal region of the nasal cavity. For example, if fluid flows from the cranial region of the nasal cavity toward the caudal region of the nasal cavity (e.g., due to gravity, a pressure gradient, and the like), blockage 108h, blockage 124h, blockage 174h, and blockage 178h can substantially prevent fluid from flowing beyond each blockage with respect to the direction of fluid flow. In the embodiment shown, blockages 108h and 124h are each disposed between the septum and the inferior nasal turbinate of different nostrils, and blockages 174h and 178h are each disposed between the septum and the middle nasal turbinate of different nostrils. However, in other embodiments, blockages 108h and 174h are each disposed between septum and the inferior nasal turbinate or between the septum and the middle nasal turbinate of the same nostril (e.g., in a stacked configuration). Similarly, in some embodiments, blockages 124h and 178h are each disposed between septum and the inferior nasal turbinate or between the septum and the middle nasal turbinate of the same nostril (e.g., in a stacked configuration). In some embodiments, blockage 108h, blockage 124h, blockage 174h, and/blockage 178h extend to the choana, whether the blockages are disposed between the septum and the middle nasal turbinate or the septum and the inferior nasal turbinate. Blockages 108h, 124h, 174h, and 178h and tubes 116h and 160h can each be inserted through either nostril of a subject and in any of the described configurations, and none are required to be in the same nostril at the same time. Further, device 100h, blockages 108h, 124h, 174h, and 178h, and tubes 116h and 160h can comprise and/or be coupled to any of the features and components described with respect to the figures and embodiments of this disclosure.

In the embodiment shown in FIG. 8, as with any of the other embodiments described in this disclosure, pump 104h can comprise more than one pump. For example, in some embodiments, tube 116h and tube 160h can each be coupled to a separate pump (e.g., tube 116h can be coupled to a first pump and tube 160h can be coupled to a second pump) to, for example, increase the ability to monitor/adjust the amount of fluid moving through each tube, to monitor/adjust the pressure at any point along each tube, and to monitor/address differences in resistance to flow inside the nasal cavity (e.g., due to the nasal valve) that may affect a desired fluid distribution through the tubes or in the nasal cavity.

In the embodiment shown in FIG. 8, blockages 108h, 124h, 174h, and 178h, are configured to accommodate tissue and/or structures within the nasal cavity. In the embodiment shown, each of blockages 108h, 124h, 174h, and 178h comprise a substantially semi-cylindrical configuration that is substantially inverse to the shape of a turbinate with which each blockage is in contact. For example, the substantially semi-cylindrical configuration of blockages 108h and 124h permit a portion of each blockage to be disposed between the septum and the inferior nasal turbinate and a portion of each blockage to extend into the inferior nasal meatus. Similarly, the substantially semi-cylindrical configuration of blockages 174h and 178h permit a portion of each blockage to be disposed between the septum and the middle nasal turbinate and a portion of each blockage to extend into the middle nasal meatus.

In the embodiment shown in FIG. 8, device 100h is configured to pump fluid into the nasal cavity of the subject to cool the brain of the subject. In some embodiments, device 100h is configured to cool the brain of the subject until a brain to body temperature gradient of at least 1° C. is achieved (e.g., a temperature gradient of 1° C., 2° C., 3° C., 4° C., 5° C., 6° C., 7° C., 8° C., 9° C., 10° C., or more). In some embodiments, device 100h is configured to cool the brain of the subject with fluid that is cooler than room temperature (e.g., cooler than 28° C., 26° C., 24° C., 22° C., 20° C., 18° C., or cooler). In some embodiments, device 100h is configured to cool the brain of the subject for at least one hour (e.g., 1 hour, 2 hours, 3 hours, 4 hours, 5 hours, 6 hours, 7 hours, 8 hours, 9 hours, 10 hour, or more). In some embodiments, device 100h is configured to cool the brain of the subject with at least 250 milliliters of fluid (e.g., 250 milliliters, 500 milliliters, 750 milliliters, 1 liter, 1.25 liters, 1.5 liters, 1.75 liters, 2 liters, or more).

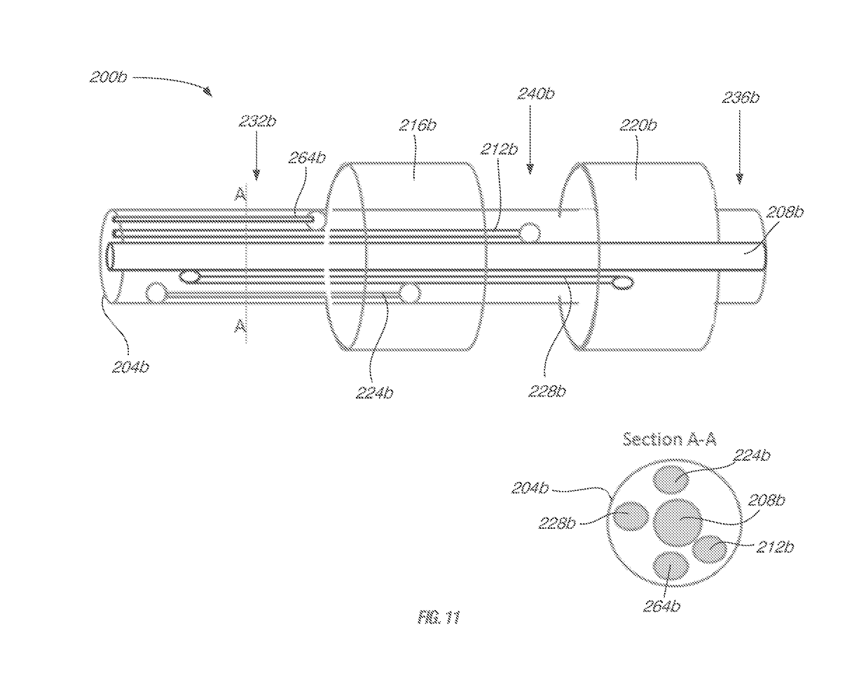

Referring now to FIG. 9, designated by reference numeral 100i is another embodiment of the present cooling devices. As in FIG. 3, cooling device 100i comprises pump 104i. Sphenoid sinus tube 128i is coupled to pump 104i and configured to be inserted through a nostril of a subject and into the sphenoid sinus of the subject such that pump 104i can, if activated, pump fluid through sphenoid sinus tube 128i and into the sphenoid sinus. Device 100i further comprises sphenoid sinus tube 182i that is coupled to pump 104i and configured to be inserted through a nostril of a subject and into the sphenoid sinus of the subject such that pump 104i can, if activated, pump fluid through sphenoid sinus tube 128i and into the sphenoid sinus. Sphenoid sinus tubes 128i and 182i can comprise one opening or a plurality of openings through which fluid can exit each sphenoid sinus tube.

A sufficient volume of fluid can be delivered through sphenoid sinus tubes 128i and 182i to both sphenoid sinuses of a subject such that fluid substantially fills the sphenoid sinus. In some embodiments, fluid can passively flow out of the sphenoid ostium of each sphenoid sinus. In some embodiments, cooling device 100i can comprise a third and fourth sphenoid sinus tube configured to be inserted through a nostril of a subject and into each sphenoid sinus of the subject. In such an embodiment, device 100i can be configured to remove (e.g., with a suctioning device) fluid from each sphenoid sinus through the third and fourth sphenoid sinus tube. In another embodiment, sphenoid sinus tube 128i and sphenoid sinus tube 182i can each comprise a multi-lumen catheter such that pump 104i can, if activated, pump fluid through a first lumen of sphenoid sinus tube 128i and through a first lumen of sphenoid sinus tube 182i and into each sphenoid sinus, and fluid can be removed from each sphenoid sinus through a second lumen of sphenoid sinus tube 128i and a second lumen of sphenoid sinus tube 182i. Sphenoid sinus tubes 128i and 182i can comprise any of the materials or configurations as described with respect to sphenoid sinus tube 128c in FIG. 3. Further, device 100i, sphenoid sinus tube 128i, and sphenoid sinus tube 182i can comprise and/or be coupled to any of the features and components described with respect to the figures and embodiments of this disclosure.

In the embodiment shown in FIG. 9, device 100i is configured to pump fluid into the sphenoid sinus of the subject to cool the brain of the subject. In some embodiments, device 100i is configured to cool the brain of the subject until a brain to body temperature gradient of at least 1° C. is achieved (e.g., a temperature gradient of 1° C., 2° C., 3° C., 4° C., 5° C., 6° C., 7° C., 8° C., 9° C., 10° C., or more). In some embodiments, device 100i is configured to cool the brain of the subject with fluid that is cooler than room temperature (e.g., cooler than 28° C., 26° C., 24° C., 22° C., 20° C., 18° C., or cooler). In some embodiments, device 100i is configured to cool the brain of the subject for at least one hour (e.g., 1 hour, 2 hours, 3 hours, 4 hours, 5 hours, 6 hours, 7 hours, 8 hours, 9 hours, 10 hour, or more). In some embodiments, device 100i is configured to cool the brain of the subject with at least 250 milliliters of fluid (e.g., 250 milliliters, 500 milliliters, 750 milliliters, 1 liter, 1.25 liters, 1.5 liters, 1.75 liters, 2 liters, or more).

FIGS. 10-13 depict another device that can be used in cooling the brain of a subject to, for example, substantially prevent fluid from entering the lungs of a patient. In the embodiment shown, device 200a-d comprises multi-lumen catheter 204a-d configured (e.g., sized) to be inserted through the mouth of a subject and into the trachea of the subject. In the embodiment shown, multi-lumen catheter 204a-d comprises first lumen 208a-d and second lumen 212a-d. First lumen 208a-d is configured such that, if multi-lumen catheter 204a-d is inserted into the trachea of a subject, first lumen 208a-d is in fluid communication with the lungs of the subject.

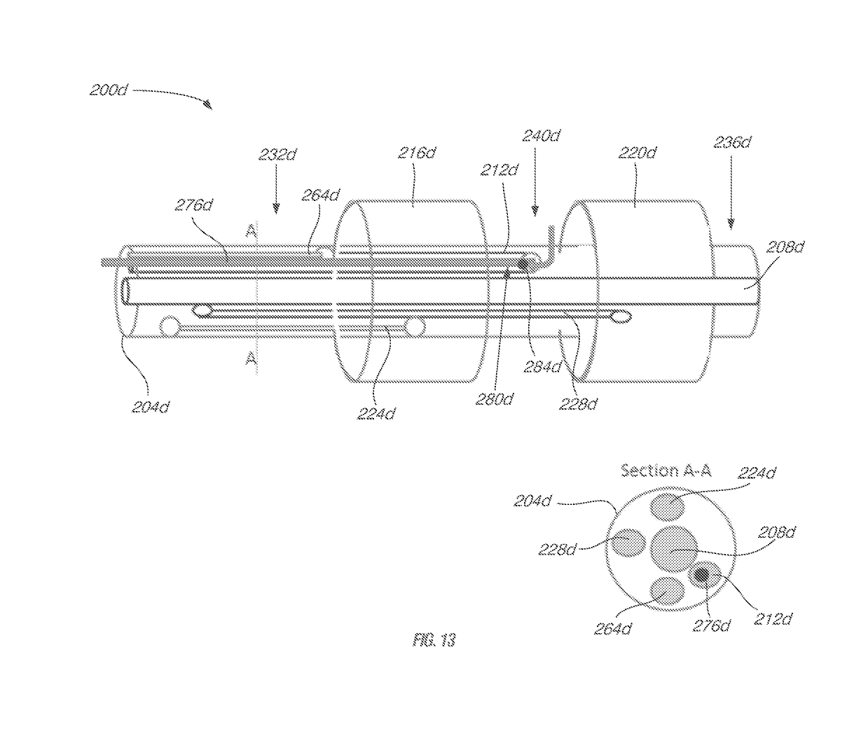

Device 200a-d further comprises inflatable cuff 216a-d that is configured to be inserted through the mouth of a subject and into the trachea of the subject. Inflatable cuff 216a-d is configured such that, if inflatable cuff 216a-d is inserted into the trachea of a subject and inflated, inflatable cuff 216a-d substantially prevents fluid from entering the lungs of the subject. Device 200a-d also comprises inflatable cuff 220a-d that is configured to be inserted through the mouth of a subject and into the trachea of the subject. In the embodiment shown, if inflatable cuff 220a-d is inserted into the trachea of a subject, inflatable cuff 220a-d is configured to be disposed distal to inflatable cuff 216a-d (e.g., closer to the lungs of the subject than inflatable cuff 216a-d). Inflatable cuff 220a-d is configured such that, if inflatable cuff 220a-d is inserted into the trachea of a subject and inflated, inflatable cuff 220a-d substantially prevents fluid from entering the lungs of the subject. Inflatable cuffs 216a-d and 220a-d can be coupled to or unitary with (e.g., formed of the same piece of material) multi-lumen catheter 204a-d. In the embodiment shown, lumen 224a-d is in fluid communication with inflatable cuff 216a-d and permits inflatable cuff 216a-d to be inflated (e.g., by a pump coupled to lumen 224a-d and/or multi-lumen catheter 204a-d). In some embodiments, pressure in inflatable cuff 216a-d can be detected by placing a sensor interior to inflatable cuff 216a-d and/or in or near lumen 224a-d (as in FIG. 12). Similarly, lumen 228a-d is in fluid communication with inflatable cuff 220a-d and permits inflatable cuff 220a-d to be inflated (e.g., by a pump coupled to lumen 228a-d and/or multi-lumen catheter 204a-d). In some embodiments, pressure in inflatable cuff 220a-d can be detected by placing a sensor interior to inflatable cuff 220a-d and/or in or near lumen 228a-d (as in FIG. 12). In some embodiments, an inflatable cuff can be inflated with a single-phase fluid (e.g., air, water, and the like). In other embodiments, an inflatable cuff can comprise double-inflatable cuffs. In still other embodiments, an inflatable cuff can be foam-filled. In such a configuration, the inflatable cuff can be introduced into the trachea in a collapsed (e.g., vacuumed) configuration and permitted to be inflated after being disposed in the trachea.

In the embodiments shown in FIGS. 10-13, inflatable cuff 216a-d and inflatable cuff 220 define three regions: first region 232a-d proximal to inflatable cuff 216a-d (e.g., between inflatable cuff 216a-d and the mouth of a subject), second region 236a-d distal to inflatable cuff 220a-d (e.g., between inflatable cuff 220a-d and the lungs of a subject), and buffer region 240a-d between first inflatable cuff 216a-d and second inflatable cuff 220a-d. Second lumen 212a-d of multi-lumen catheter 204a-d is in fluid communication with buffer region 240a-d such that pressure in buffer region 240a-d can be increased and decreased. For example, inflatable cuff 216a-d is subject to atmospheric pressure and/or hydrostatic pressure (e.g., from fluid in the aerodigestive tract of a subject). Pressure in buffer region 240a-d can be increased (e.g., by activating device 200a-d (e.g., via a pump)) such that the pressure in buffer region 240a-d is equal to or greater than pressure in first region 232a-d (e.g., the atmospheric pressure and hydrostatic pressure at or near inflatable cuff 216a-d) to discourage fluid from moving past inflatable cuff 216a-d toward the lungs of a subject. In some embodiments, a positive pressure can be introduced to buffer region 240a-d such that buffer region 240a-d has a pressure of at least 10 cmH₂O (e.g., 10 cmH₂O, 15 cmH₂O, 20 cmH₂O, 25 cmH₂O, 30 cmH₂O).

In the embodiment shown in FIG. 10, device 200a further comprises sensor 252a configured to detect at least one of pressure, temperature, and impedance in first region 232a. Sensor 252a can be coupled to, for example, inflatable cuff 216a and/or multi-lumen catheter 204a such that sensor 252a can detect pressure, temperature, and/or impedance at or near inflatable cuff 216a. In the embodiment shown in FIG. 10, device 200a further comprises sensor 256a configured to detect at least one of pressure, temperature, and impedance in buffer region 240a. Sensor 256a can be coupled to lumen 212a of multi-lumen catheter 204a as depicted. In other embodiments, sensor 256a can be coupled to another portion of multi-lumen catheter 204a, inflatable cuff 216a, and/or inflatable cuff 220a. Device 200a further comprises sensor 260a configured to detect at least one of pressure, temperature, and impedance in second region 236a (e.g., pressure, temperature, and/or impedance at or near inflatable cuff 220a (e.g., the pressure in the lungs)). Sensor 260a can be coupled to lumen 208a of multi-lumen catheter 204a, some other portion of multi-lumen catheter 204a, and/or inflatable cuff 220a. Any sensor can be disposed exterior to or interior to a lumen. Any connections (e.g., electrical) to a sensor can be disposed in a lumen.

Sensors 252a, 256a and 260a can be used to determine a pressure to introduce to buffer region 240a. For example, if sensor 252a detects a given pressure, pressure can be introduced to buffer region 240a through second lumen 212a of multi-lumen catheter 204a until sensor 256a detects a higher pressure than that detected by sensor 252a.

Sensors 252a, 256a and 260a can also be used to determine if inflatable cuff 216a and/or inflatable cuff 220a has failed. For example, after a positive pressure has been introduced to buffer region 240a, if sensor 252a and 256a detect the same or substantially similar pressure, inflatable cuff 216a may have failed (and, thus, fluid may have entered buffer region 240a). Similarly, if sensor 256a and 260a detect the same or substantially similar pressure, inflatable cuff 220a may have failed (and, thus, fluid may have entered second region 236a). As another example, after a positive pressure has been introduced to buffer region 240a, if sensor 252a and 256a detect the same or substantially similar temperature, inflatable cuff 216a may have failed (and, thus, fluid may have entered buffer region 240a). Similarly, if sensor 256a and 260a detect the same or substantially similar temperature, inflatable cuff 220a may have failed (and, thus, fluid may have entered second region 236a). As yet another example, after a positive pressure has been introduced to buffer region 240a, if sensor 252a and 256a detect the same or substantially similar impedance, inflatable cuff 216a may have failed (and, thus, fluid may have entered buffer region 240a). Similarly, if sensor 256a and 260a detect the same or substantially similar impedance, inflatable cuff 220a may have failed (and, thus, fluid may have entered second region 236a).

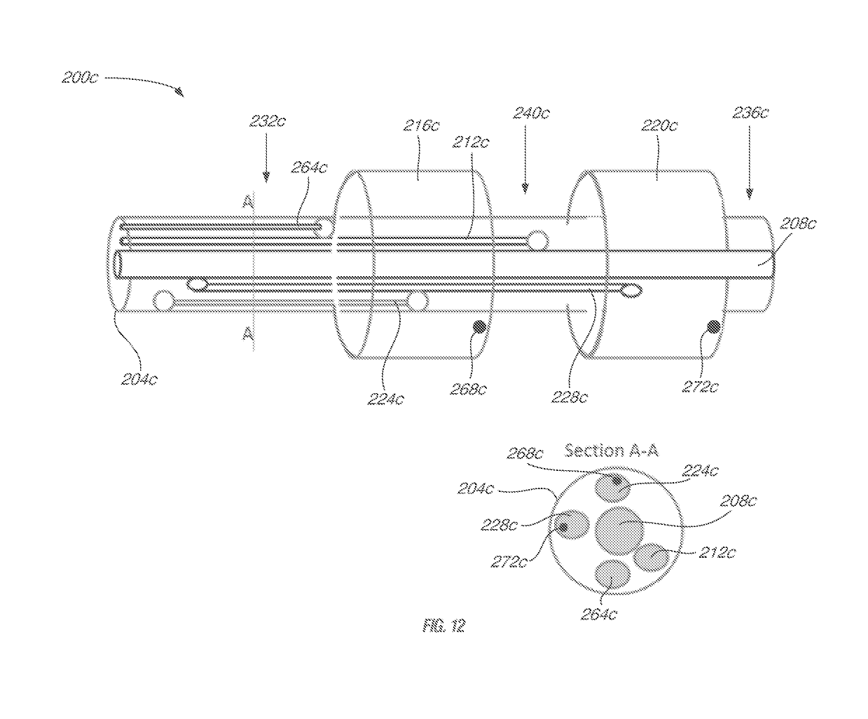

In the embodiment shown in FIG. 11-13, multi-lumen catheter 204b-d comprises third lumen 264b-d in fluid communication with first region 232b-d (e.g., at or near inflatable cuff 216b-d) such that pressure in first region 232b-d can be increased and decreased. For example, pressure in first region 232b-d can be decreased (e.g., by activating device 200b-d (e.g., via a pump) to remove fluid near inflatable cuff 216b-d) such that the pressure in first region 232b-d is equal to or less than the pressure introduced into buffer region 240b-d to discourage fluid from moving past inflatable cuff 216b-d toward the lungs of a subject.

In the embodiment shown in FIG. 12, device 200c further comprises cuff sensor 268c disposed interior to inflatable cuff 216c and cuff sensor 272c disposed interior to inflatable cuff 220c. Each of cuff sensors 268c and 272c can be disposed at (e.g., coupled to) the distal end of the respective inflatable cuff or wherever fluid would likely accumulate due to gravity. Cuff sensor 268c is configured to detect if pressure in inflatable cuff 216c decreases (and, thus, if fluid potentially moves past inflatable cuff 216c toward the lungs of the subject); and, similarly, cuff sensor 272c is configured to detect if pressure in inflatable cuff 220c decreases (and, thus, if fluid potentially moves past inflatable cuff 220c toward the lungs of the subject). In some embodiments, cuff sensor 268c and cuff sensor 272c can further detect if pressure in inflatable cuff 216c and inflatable cuff 220c, respectively, increases.

In the embodiment shown in FIG. 13, device 200d further comprises sump line 276d disposed (e.g., removably) in lumen 212d of multi-lumen catheter 204d such that sump line 276d is in fluid communication with buffer region 240d. For example, sump line 276d can be inserted into multi-lumen catheter 204d (e.g., lumen 212d) after multi-lumen catheter 204d is inserted into the trachea of a subject. Device 200d is configured is configured such that, if activated, device 200d can remove fluid from buffer region 240d through sump line 276d (e.g., if fluid is able to move past inflatable cuff 216d into buffer region 240d). In some embodiments, first end 280d of sump line 276d can comprise a bevel cut (e.g., to reduce a chance of suction trauma to the trachea). First end 280d of sump line 276d can be positioned near the distal end of buffer region 240d or wherever fluid would likely accumulate due to, for example, gravity or any positive pressure. In such an embodiment, at least a portion of lumen 212d may comprise a large diameter to accommodate sump line 276d and such that sump line 276d can remove fluid from buffer region 240d while minimizing a drop in pressure in buffer region 240d. In the embodiment shown in FIG. 13, sump sensor 284d can be disposed at or near first end 280d of sump line 276 (e.g., sump sensor 284d can be coupled to first end 280d of sump line 276, as shown), and device 200d can be configured to activate if sump sensor 284d detects fluid in buffer region 240d. For example, sump sensor 284d can be conductance or impedance-based. In other embodiments, sump sensor 284d can be configured to detect a marker (e.g., saline) in fluid circulated through the aerodigestive tract.

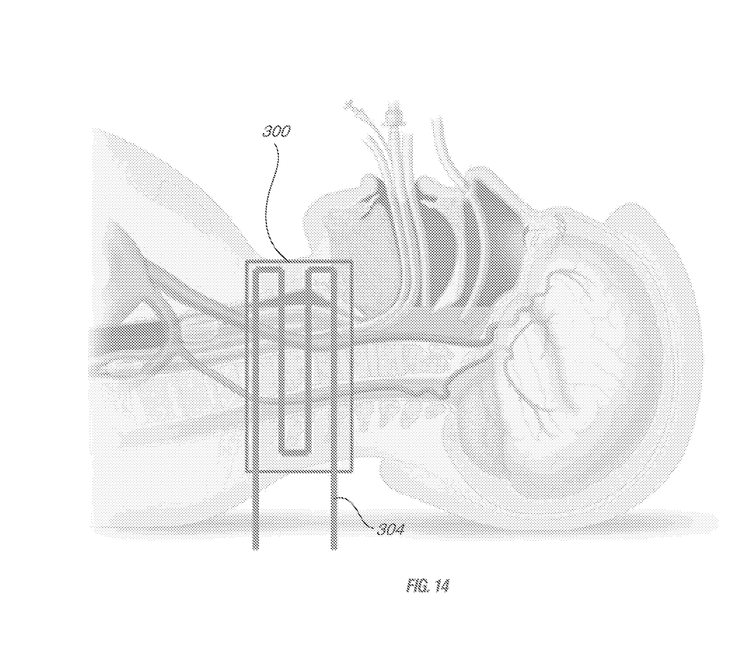

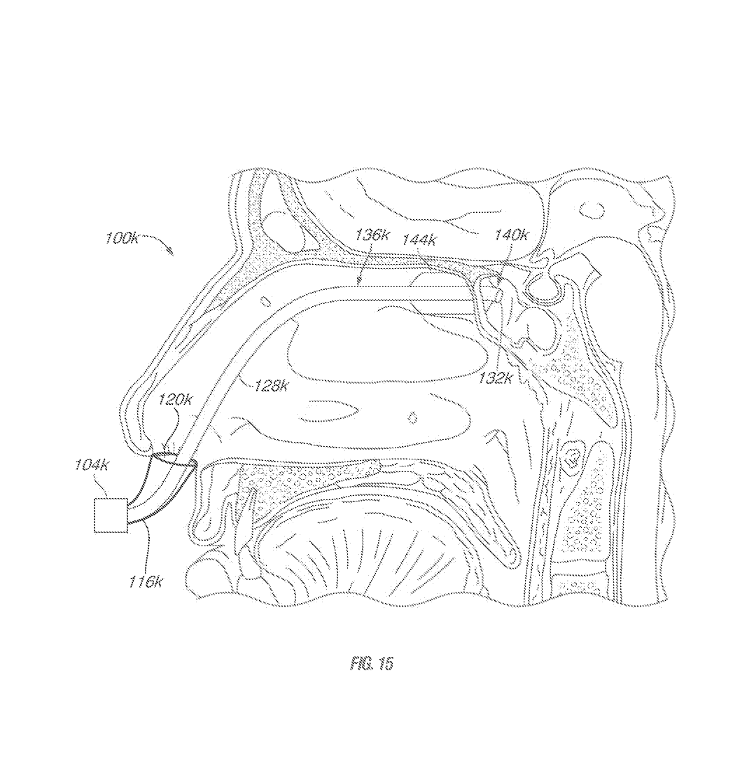

Some embodiments of the present cooling devices and methods can further comprise cooling collar 300, as depicted in FIG. 14. Cooling collar 300 can be wrapped at least partially around the neck of a subject. Cooling fluid can then be circulated through tube 304. to provide further cooling to the brain of the subject.

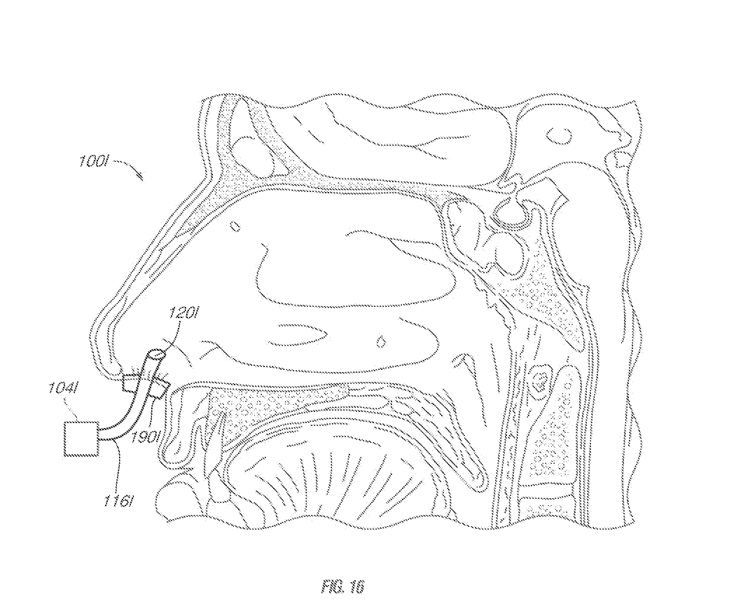

Referring now to FIG. 15, designated by reference numeral 100k is another embodiment of the present cooling devices. Device 100k comprises pump 104k and tube 116k configured to be disposed adjacent to or interior to a nostril of a subject. Such a configuration can, for example, assist in preventing trauma to the nasal cavity, such as abrasions and associated bleeding (which can be exacerbated by anticoagulants). Tube 116k can comprise substantially the same shape and/or diameter of a nostril and can be disposed such that tube 116k is adjacent to the nostril of a subject, as depicted in FIG. 15, to prevent fluid from exiting the nostril. In some embodiments, tube 116k can be inserted into a nostril a suitable distance to, for example, prevent fluid from exiting the nostril and to prevent tube 116k from exiting the nostril. Tube 116k is coupled to pump 104k such that pump 104k can, if activated, pump fluid through tube 116k and out of opening 120k (e.g., toward the cranial region of the nasal cavity). In the embodiment shown in FIG. 15, tube 116k comprises one opening through which fluid can exit tube 116k. In other embodiments, tube 116k can comprise a plurality of openings through which fluid can exit tube 116k.

In some embodiments, tube 116k and pump 104k independently comprise a cooling device. However, in the embodiment shown in FIG. 15, cooling device 100k further comprises sphenoid sinus tube 128k that is substantially coaxial with tube 116k. In other embodiments, sphenoid sinus tube 128k is not substantially coaxial with tube 116k. In still other embodiments, cooling device 100k may comprise another tube described in this disclosure. Sphenoid sinus tube 128k is coupled to pump 104k and configured to be inserted through either nostril of a subject and into a respective sphenoid sinus of the subject such that pump 104k can, if activated, pump fluid through sphenoid sinus tube 128k and into the respective sphenoid sinus. As discussed with respect to FIG. 5, in some embodiments, sphenoid sinus tube 128k can be coupled to pump 104k (e.g., a roller pump, a wall suction device, and the like) and configured to be inserted through a nostril of a subject and into a respective sphenoid sinus of the subject such that pump 104k can, if activated, pump fluid out of the sphenoid sinus (e.g., creating negative pressure inside the sphenoid sinus) through sphenoid sinus tube 128k. In the embodiment shown in FIG. 15, sphenoid sinus tube 128k comprises one opening 132k through which fluid can exit sphenoid sinus tube 128k; however, in other embodiments, sphenoid sinus tube 128k can comprise a plurality of openings through which fluid can exit sphenoid sinus tube 128k (as depicted in other figures).

A sufficient volume of fluid can be delivered to a sphenoid sinus of a subject such that fluid substantially fills the sphenoid sinus. In some embodiments, fluid can passively flow out of the sphenoid ostium of the sphenoid sinus in which sphenoid sinus tube 128k is inserted. In some embodiments, where the sphenoid sinus associated with a first nostril is in fluid communication with the sphenoid sinus associated with a second nostril, fluid can passively flow out of either sphenoid ostium. In some embodiments, cooling device 100k can comprise a second sphenoid sinus tube configured to be inserted through a nostril of a subject and into a sphenoid sinus of the subject. In such an embodiment, device 100k can be configured to remove (e.g., with a suctioning device) fluid from the sphenoid sinus through the second sphenoid sinus tube. In some embodiments, where the sphenoid sinus associated with a first nostril is in fluid communication with the sphenoid sinus associated with a second nostril, fluid can be actively removed from either sphenoid sinus through the second tube. In another embodiment, sphenoid sinus tube 128k can comprise a multi-lumen catheter such that pump 104k can, if activated, pump fluid through a first lumen of sphenoid sinus tube 128k and into the sphenoid sinus, and fluid can be removed from the sphenoid sinus through a second lumen of sphenoid sinus tube 128k.

Any tube in this application can comprise any suitable, biocompatible material, whether the tube is directed toward the cranial region of the nasal cavity or inserted into the sphenoid sinus or otherwise placed in and/or adjacent to the nose. For example, in some embodiments, sphenoid sinus tube 128k comprises a substantially malleable material. In some embodiments, sphenoid sinus tube 128k comprises a substantially rigid material. In other embodiments, sphenoid sinus tube 128k comprises first portion 136k, which comprises a substantially rigid material and second portion 140k, which comprises a substantially malleable material. In some embodiments, sphenoid sinus tube 128k can comprise a friction-reducing coating, such as Teflon, to enable sphenoid sinus tube 128k to be more easily inserted through a sphenoid ostium.

Sphenoid sinus tube 128k can comprise or be coupled to various other components. For example, sphenoid sinus tube 128k can be coupled to a pressure sensor that is configured to detect if pressure in the sphenoid sinus meets or exceeds a threshold pressure (e.g., to prevent damage to tissue and/or structures defining the sphenoid sinus). If pressure in the sphenoid sinus meets or exceeds a threshold pressure, the cooling device (e.g., cooling device 100k) can, for example, reduce the flow rate of fluid through sphenoid sinus tube 128k (e.g., automatically or by user input). Sphenoid sinus tube 128k can also comprise or be coupled to a stopper configured to limit the extent to which sphenoid sinus tube 128k can enter the sphenoid sinus (e.g., to prevent damage to tissue and/or structures defining the sphenoid sinus).

As depicted and discussed in other embodiments, sphenoid sinus tube 128k can also be configured such that, after sphenoid sinus tube 128k is inserted into the sphenoid sinus of the subject, a portion of sphenoid sinus tube 128k prevents sphenoid sinus tube 128k from exiting the sphenoid sinus.

Further, as depicted in FIG. 15, sphenoid sinus tube 128k also can comprise (or be coupled to) inflatable cuff 144k. Inflatable cuff 144k is positioned such that, after sphenoid sinus tube 128k is inserted into the sphenoid sinus of the subject, inflatable cuff 144k can be inflated (in some instances, temporarily, for example, prior to fluid entering the sphenoid sinus) to engage tissue defining a sphenoid ostium and, therefore, increasing the area of that sphenoid ostium. In some embodiments, inflatable cuff 144k is inflated to increase the area of a sphenoid ostium, and then deflated prior to introducing fluid through sphenoid sinus tube 128k into the sphenoid sinus (e.g., to permit fluid to passively flow out of the sphenoid sinus). In other embodiments, a sphenoid ostium can be blocked or partially blocked (e.g., by inflating or partially inflating inflatable cuff 144k or by some other blockage) while fluid is introduced through sphenoid sinus tube 128k into the sphenoid sinus (e.g., to increase a volume of fluid in the sphenoid sinus and, thus, increase the surface area of the sphenoid sinus in contact with the fluid). Further, device 100k, tube 116k, and sphenoid sinus tube 116k can comprise and/or be coupled to any of the features and components described with respect to the figures and embodiments of this disclosure. For example, this embodiment can be combined with embodiments disclosing delivery of fluid into areas internal to the nasal valve, such as into the nasal cavity, sinuses, nasopharynx, and/or a combination thereof. Similarly, this embodiment can be combined with embodiments disclosing removal of fluid from areas internal to the nasal valve, such as from the nasal cavity, sinuses, nasopharynx, and/or combinations thereof. The tubes delivering or removing fluid from areas internal to the nasal valve can be substantially coaxial and/or parallel to the fluid delivery tube of this embodiment such that one tube passes through another tube.

In the embodiment shown in FIG. 15, device 100k is configured to pump fluid into the nasal cavity of the subject to cool the brain of the subject. In some embodiments, device 100k is configured to cool the brain of the subject until a brain to body temperature gradient of at least 1° C. is achieved (e.g., a temperature gradient of 1° C., 2° C., 3° C., 4° C., 5° C., 6° C., 7° C., 8° C., 9° C., 10° C., or more). In some embodiments, device 100k is configured to cool the brain of the subject with fluid that is cooler than room temperature (e.g., cooler than 28° C., 26° C., 24° C., 22° C., 20° C., 18° C., or cooler). In some embodiments, device 100k is configured to cool the brain of the subject for at least one hour (e.g., 1 hour, 2 hours, 3 hours, 4 hours, 5 hours, 6 hours, 7 hours, 8 hours, 9 hours, 10 hour, or more). In some embodiments, device 100k is configured to cool the brain of the subject with at least 250 milliliters of fluid (e.g., 250 milliliters, 500 milliliters, 750 milliliters, 1 liter, 1.25 liters, 1.5 liters, 1.75 liters, 2 liters, or more).

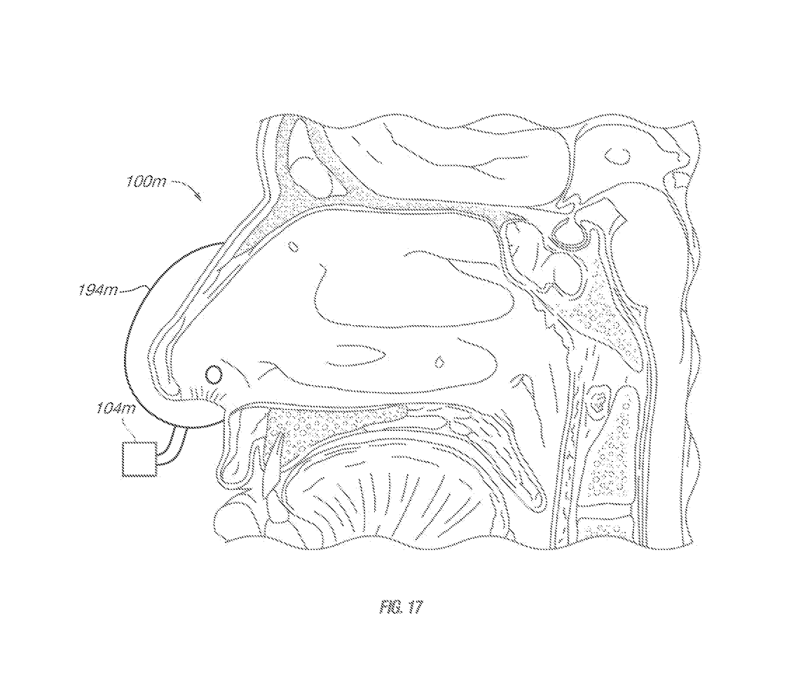

Referring now to FIG. 16, designated by reference numeral 100l is another embodiment of the present cooling devices. Device 100l comprises pump 104l and tube 116l configured to be disposed in a nostril of a subject. Such a configuration can, for example, assist in preventing trauma to the nasal cavity, such as abrasions and associated bleeding (which can be exacerbated by anticoagulants). In the embodiment shown, tube 116l is inserted into the nostril of a subject and terminates prior to the nasal valve. In other embodiments, tube 116l can be inserted into the nostril of a subject such that it is adjacent to or past the nasal valve. Tube 116l is coupled to pump 104l such that pump 104l can, if activated, pump fluid through tube 116l and out of opening 120l (e.g., toward the cranial region of the nasal cavity). In the embodiment shown in FIG. 16, tube 116l comprises one opening through which fluid can exit tube 116l. In other embodiments, tube 116l can comprise a plurality of openings through which fluid can exit tube 116l. In the embodiment shown, device 100l further comprises stopper 190l (e.g., adjacent to the nares or peri-nares region), which is configured to prevent fluid from exiting the nostril of a subject. Stopper 1901 can comprise substantially the same diameter and/or shape of a nostril of a subject and/or can be made of material configured to conform to the shape of a nostril of a subject. Stopper 190l can be disposed outside the nasal vestibule, inside the nasal vestibule, or both outside and inside the nasal vestibule. Device 100l, tube 116l, and stopper 190l can comprise and/or be coupled to any of the features and components described with respect to the figures and embodiments of this disclosure. For example, this embodiment can be combined with embodiments disclosing delivery of fluid into areas internal to the nasal valve, such as into the nasal cavity, sinuses, nasopharynx, and/or a combination thereof. Similarly, this embodiment can be combined with embodiments disclosing removal of fluid from areas internal to the nasal valve, such as from the nasal cavity, sinuses, nasopharynx, and/or combinations thereof. The tubes delivering or removing fluid from areas internal to the nasal valve can be substantially coaxial and/or parallel to the fluid delivery tube of this embodiment such that one tube passes through another tube.

In the embodiment shown in FIG. 16, device 100l is configured to pump fluid into the nasal cavity of the subject to cool the brain of the subject. In some embodiments, device 100l is configured to cool the brain of the subject until a brain to body temperature gradient of at least 1° C. is achieved (e.g., a temperature gradient of 1° C., 2° C., 3° C., 4° C., 5° C., 6° C., 7° C., 8° C., 9° C., 10° C., or more). In some embodiments, device 100l is configured to cool the brain of the subject with fluid that is cooler than room temperature (e.g., cooler than 28° C., 26° C., 24° C., 22° C., 20° C., 18° C., or cooler). In some embodiments, device 100l is configured to cool the brain of the subject for at least one hour (e.g., 1 hour, 2 hours, 3 hours, 4 hours, 5 hours, 6 hours, 7 hours, 8 hours, 9 hours, 10 hour, or more). In some embodiments, device 100l is configured to cool the brain of the subject with at least 250 milliliters of fluid (e.g., 250 milliliters, 500 milliliters, 750 milliliters, 1 liter, 1.25 liters, 1.5 liters, 1.75 liters, 2 liters, or more).

Referring now to FIG. 17, designated by reference numeral 100m is another embodiment of the present cooling devices. Device 100m comprises pump 104m and nasal mask 194m disposed about the nose of a subject. Nasal mask 194m can be suitably sealed with respect to the subject's head (e.g., by tightening bands around the head of a subject) to prevent fluid from exiting nasal mask 194m. Such a configuration can, for example, assist in preventing trauma to the nasal cavity, such as abrasions and associated bleeding (which can be exacerbated by anticoagulants). Nasal mask 194m is coupled to pump 104m such that pump 104m can, if activated, pump fluid into nasal mask 194m such that fluid can enter the nose of a subject. Device 100m and nasal mask 194m can comprise and/or be coupled to any of the features and components described with respect to the figures and embodiments of this disclosure. As another example, this embodiment can be combined with embodiments disclosing delivery of fluid into areas internal to the nasal valve, such as into the nasal cavity, sinuses, nasopharynx, and/or a combination thereof. Similarly, this embodiment can be combined with embodiments disclosing removal of fluid from areas internal to the nasal valve, such as from the nasal cavity, sinuses, nasopharynx, and/or combinations thereof. The tubes delivering or removing fluid from areas internal to the nasal valve can be substantially coaxial and/or parallel to the fluid delivery tube of this embodiment such that one tube passes through another tube.

In the embodiment shown in FIG. 17, device 100m is configured to pump fluid into the nasal cavity of the subject to cool the brain of the subject. In some embodiments, device 100m is configured to cool the brain of the subject until a brain to body temperature gradient of at least 1° C. is achieved (e.g., a temperature gradient of 1° C., 2° C., 3° C., 4° C., 5° C., 6° C., 7° C., 8° C., 9° C., 10° C., or more). In some embodiments, device 100m is configured to cool the brain of the subject with fluid that is cooler than room temperature (e.g., cooler than 28° C., 26° C., 24° C., 22° C., 20° C., 18° C., or cooler). In some embodiments, device 100m is configured to cool the brain of the subject for at least one hour (e.g., 1 hour, 2 hours, 3 hours, 4 hours, 5 hours, 6 hours, 7 hours, 8 hours, 9 hours, 10 hour, or more). In some embodiments, device 100m is configured to cool the brain of the subject with at least 250 milliliters of fluid (e.g., 250 milliliters, 500 milliliters, 750 milliliters, 1 liter, 1.25 liters, 1.5 liters, 1.75 liters, 2 liters, or more).

The present disclosure also includes methods for cooling the brain. Some embodiments of the present cooling methods comprise inserting a first guide-wire through a nostril of a subject and into a sphenoid sinus of the subject; inserting a first tube (e.g., sphenoid sinus tube 128c and/or sphenoid sinus tube 128i) over the first guide-wire and into the sphenoid sinus; removing the first guide-wire; and circulating fluid through the first tube and into the sphenoid sinus to cool the brain of the subject.

Some embodiments of the present methods comprise inserting a first guide-wire through a nostril of a subject and into a sphenoid sinus of the subject; inserting a first tube (e.g., sphenoid sinus tube 128c and/or sphenoid sinus tube 128i) over the first guide-wire and into the sphenoid sinus; removing the first guide-wire; circulating fluid through the nasal cavity; and removing fluid from the sphenoid sinus through the first tube to cool the brain of the subject.

Some embodiments further comprise increasing the area of a sphenoid ostium to cool the mucosa of the subject. In some embodiments, increasing the area of a sphenoid ostium comprises inflating an inflatable cuff (e.g., inflatable cuff 144c) such that the inflatable cuff engages (in some instances, temporarily, for example, prior to fluid entering the sphenoid sinus) tissue defining the sphenoid ostium. Some embodiments comprise inserting a second tube through a nostril of a subject and into a sphenoid sinus of the subject (e.g., a separate tube or, if they are multi-lumen catheters, sphenoid sinus tubes 128c and/or 128i); and removing fluid from the sphenoid sinus through the second tube. In some embodiments, the first tube comprises a multi-lumen catheter such that fluid can be circulated through a first lumen of the multi-lumen catheter, the method further comprising removing fluid from the sphenoid sinus through a second lumen of the multi-lumen catheter. In some embodiments, fluid is circulated through the first tube at a rate of 1-500 ml/min.

Some embodiments of the present methods further comprise inserting a third tube (e.g., sphenoid sinus tube 182i) through a nostril of a subject and into the sphenoid sinus of the subject; and circulating fluid through the third tube and into the sphenoid sinus to cool the brain of the subject. Some embodiments comprise inserting a fourth tube through a nostril of a subject and into a sphenoid sinus (e.g., a separate tube or, if it is a multi-lumen catheter, sphenoid sinus tube 182i); and removing fluid from the sphenoid sinus through the fourth tube. In some embodiments, the third tube comprises a multi-lumen catheter such that fluid can be circulated through a first lumen of the multi-lumen catheter, the method further comprising removing fluid from the sphenoid sinus through a second lumen of the multi-lumen catheter.

Some embodiments of the present methods further comprise, prior to inserting a first tube over the first guide-wire, inserting an inflatable cuff (e.g., inflatable cuff 144c) over the first guide-wire; and inflating the inflatable cuff such that the inflatable cuff engages a sphenoid ostium. Some embodiments further comprise deflating (or partially deflating) the inflatable cuff. Some embodiments further comprise removing the inflatable cuff. In some embodiments, inflating the inflatable cuff comprises inflating the inflatable cuff such that adjacent mucosa, connective tissue, and/or bone is displaced. Some embodiments further comprise deflating (or partially deflating) the inflatable cuff.

Some embodiments of the present methods further comprise, prior to removing the first guide-wire, inserting a second guide-wire into the sphenoid sinus (e.g., associated with the same or a different nostril); inserting an inflatable cuff (e.g., inflatable cuff 144c) over the second guide-wire; and inflating the inflatable cuff such that the inflatable cuff engages a sphenoid ostium. Some embodiments further comprise deflating (or partially deflating) the inflatable cuff. Some embodiments further comprise removing the inflatable cuff; and removing the second guide-wire. In some embodiments, inflating the inflatable cuff comprises inflating the inflatable cuff such that adjacent mucosa, connective tissue, and/or bone is displaced. Some embodiments further comprise deflating (or partially deflating) the inflatable cuff.

Some embodiments of such methods comprise circulating fluid through the first tube and/or the second tube (e.g., sphenoid sinus tube 128c, sphenoid sinus tube 128i, and/or sphenoid sinus tube 182i) and into the sphenoid sinus until a brain to body temperature gradient of at least 1° C. is achieved (e.g., a temperature gradient of 1° C., 2° C., 3° C., 4° C., 5° C., 6° C., 7° C., 8° C., 9° C., 10° C., or more). In some embodiments, the fluid is cooler than room temperature (e.g., cooler than 28° C., 26° C., 24° C., 22° C., 20° C., 18° C., or cooler). In some embodiments, the fluid is circulated through the first tube and/or the second tube for at least one hour (e.g., 1 hour, 2 hours, 3 hours, 4 hours, 5 hours, 6 hours, 7 hours, 8 hours, 9 hours, 10 hour, or more). In some embodiments, at least 250 milliliters of fluid is circulated through the first tube and/or the second tube (e.g., 250 milliliters, 500 milliliters, 750 milliliters, 1 liter, 1.25 liters, 1.5 liters, 1.75 liters, 2 liters, or more).

Some embodiments of the present methods comprise inserting a first tube through a nostril of a subject (e.g., tubes 116a, 116b, 116e, 116g, and/or 116h); directing the first tube toward the cranial region of the nasal cavity; and decreasing resistance to cooling fluid flow in the cranial region of the nasal cavity (discussed further below) and/or increasing resistance to cooling fluid flow in the caudal region of the nasal cavity (discussed further below); and circulating fluid through the first tube into the nasal cavity to cool the brain of the subject. In some embodiments, the method comprises disposing a second tube through a nostril of a subject (e.g., tubes 160g and 160h); directing the second tube toward the cranial region of the nasal cavity; and circulating fluid through the second tube into the nasal cavity to cool the brain of the subject.

In some embodiments, increasing resistance to cooling fluid flow in the caudal region of the nasal cavity comprises inserting a first blockage through a nostril of a subject (e.g., blockages 108a, 108b, 108e, 108g, 108h, and/or nasal insert 1480; and disposing the first blockage in the caudal region of the nasal cavity. In some embodiments, the first blockage is disposed between the septum and an inferior nasal turbinate (e.g., in either nostril). In some embodiments, the first blockage is disposed between the septum and a middle nasal turbinate (e.g., in either nostril). In some embodiments, the first blockage is disposed in the inferior nasal meatus (e.g., in either nostril). In other embodiments, the first blockage is disposed in the middle nasal meatus (e.g., in either nostril).

In some embodiments, increasing resistance to cooling fluid flow in the caudal region of the nasal cavity further comprises inserting a second blockage through a nostril of a subject (e.g., blockages 124b, 124e, 124g, 124h, and/or nasal insert 1480; and disposing the second blockage in the caudal region of the nasal cavity. In some embodiments, the second blockage is disposed between the septum and an inferior nasal turbinate (e.g., in the same or a different nostril as the first blockage). In some embodiments, the second blockage is disposed between the septum and a middle nasal turbinate (e.g., in the same or a different nostril as the first blockage). In some embodiments, increasing resistance to cooling fluid flow in the caudal region of the nasal cavity further comprises inserting a third blockage through a nostril of a subject (e.g., blockage 174h); and disposing the third blockage in the caudal region of the nasal cavity. In some embodiments, the third blockage is disposed between the septum and an inferior nasal turbinate (e.g., in either nostril). In some embodiments, the third blockage is disposed between the septum and a middle nasal turbinate (e.g., in either nostril). In some embodiments, the third blockage is disposed in the inferior nasal meatus (e.g., in either nostril). In other embodiments, the third blockage is disposed in the middle nasal meatus (e.g., in either nostril).

In some embodiments, increasing resistance to cooling fluid flow in the caudal region of the nasal cavity further comprises inserting a fourth blockage through a nostril of a subject (e.g., blockage 178h); and disposing the fourth blockage in the caudal region of the nasal cavity. In some embodiments, the fourth blockage is disposed between the septum and an inferior nasal turbinate (e.g., in either nostril). In some embodiments, the fourth blockage is disposed between the septum and a middle nasal turbinate (e.g., in either nostril). In some embodiments, at least one of the first blockage and the third blockage comprises an inflatable cuff (e.g., inflatable cuff 112a), and the method further comprises inflating the inflatable cuff. In some embodiments, at least one of the second blockage and the fourth blockage comprises an inflatable cuff, and the method further comprising inflating the inflatable cuff. Though the blockages in FIG. 8 comprise a semi-circular configuration, they are not required to, and any of the above-described configurations can similarly be employed with non-semi-circular blockages.

In some embodiments, increasing resistance to cooling fluid flow in the caudal region of the nasal cavity comprises introducing a substance that is configured to increase the volume of tissue in the caudal region of the nasal cavity. The substance can comprise, for example, a vasodilator, an irritant, an allergen, a cholinergic, a particulate, nitroglycerine, an erectile dysfunction therapy, and/or a combination thereof. In some embodiments, the substance is introduced systemically; and in other embodiments, the substance is introduced to the caudal region of the nasal cavity. In some embodiments, any of tubes 116a, 116b, 116e, 116g, 116h, 128c, 128i, 160g, 160h, 182i and/or nasal insert 148f can be configured to introduce the substance (e.g., before, after, or during introducing of fluid to the nasal cavity).

In some embodiments, decreasing resistance to cooling fluid flow in the cranial region of the nasal cavity comprises inserting a blockage (e.g., a tube, a stent, and the like) in one or more cranial channels of the nasal cavity; and removing the blockage from the one or more cranial channels. In some embodiments, the blockage is self-expanding. In some embodiments, decreasing resistance to cooling fluid flow in the cranial region of the nasal cavity comprises inserting an inflatable cuff in one or more cranial channels of the nasal cavity; and inflating the inflatable cuff. Some embodiments further comprise deflating (or partially deflating) the inflatable cuff. In some embodiments, decreasing resistance to cooling fluid flow in the cranial region of the nasal cavity further comprises removing the inflatable cuff from the one or more cranial channels in the nasal cavity.

In some embodiments, decreasing resistance to cooling fluid flow in the cranial region of the nasal cavity comprises introducing a substance that is configured to decrease the volume of tissue in the cranial region of the nasal cavity. The substance can comprise, for example, an anticholinergic, an antihistamine, a mast cell inhibitor, a vasoconstrictor (e.g., cocaine, oxymetazoline, phenylephrine, xylometazoline, and naphazoline), and/or a combination thereof. In some embodiments, the substance is introduced systemically; and in other embodiments, the substance is introduced to the cranial region of the nasal cavity. In some embodiments, any of tubes 116a, 116b, 116e, 116g, 116h, 128c, 128i, 160g, 160h, 182i and/or nasal insert 148f can be configured to introduce the substance (e.g., before, after, or during introducing of fluid to the nasal cavity).

In some embodiments, the methods comprise circulating fluid through the first tube and/or the second tube (e.g., 116a, 116b, 116e, 116g, 116h, 160g, 160h, and/or nasal insert 1480; until a brain to body temperature gradient of at least 1° C. is achieved (e.g., a temperature gradient of 1° C., 2° C., 3° C., 4° C., 5° C., 6° C., 7° C., 8° C., 9° C., 10° C., or more). In some embodiments, the fluid is cooler than room temperature (e.g., cooler than 28° C., 26° C., 24° C., 22° C., 20° C., 18° C., or cooler). In some embodiments, the fluid is circulated through the first tube and/or the second tube for at least one hour (e.g., 1 hour, 2 hours, 3 hours, 4 hours, 5 hours, 6 hours, 7 hours, 8 hours, 9 hours, 10 hour, or more). In some embodiments, at least 250 milliliters of fluid is circulated through the first tube and/or the second tube (e.g., 250 milliliters, 500 milliliters, 750 milliliters, 1 liter, 1.25 liters, 1.5 liters, 1.75 liters, 2 liters, or more).

The above specification and examples provide a complete description of the structure and use of exemplary embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the present devices are not intended to be limited to the particular forms disclosed. Rather, they include all modifications and alternatives falling within the scope of the claims, and embodiments other than the one shown may include some or all of the features of the depicted embodiment. For example, components may be combined as a unitary structure and/or connections may be substituted. Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1.-144. (canceled)

145. A cooling device comprising:
a pump; and
a first nasal insert coupled to the pump such that the pump can pump fluid through the first nasal insert, the first nasal insert comprising:
a first portion configured to be inserted through a nostril of a subject and directed toward the cranial region of the nasal cavity; and
a second portion configured to be inserted through a nostril of a subject and disposed in the caudal region of the nasal cavity such that resistance to cooling fluid flow in the caudal region of the nasal cavity is increased;
where the device is configured to pump fluid into the nasal cavity of the subject to cool the brain of the subject.

146. (canceled)

147. The cooling device of claim 145, where the device is configure to cool the brain of the subject with fluid that is cooler than room temperature.

148. The cooling device of claim 145, where the device is configured to cool the brain of the subject for at least one hour.

149. The cooling device of claim 145, where the device is configured to cool the brain of the subject with at least 250 milliliters of fluid.

150. The cooling device of claim 145 further comprising:
a second nasal insert coupled to the pump such that the pump can pump fluid through the second nasal insert, the second nasal insert comprising:
a first portion configured to be inserted through a nostril of a subject and directed toward the cranial region of the nasal cavity; and
a second portion configured to be inserted through a nostril of a subject and disposed in the caudal region of the nasal cavity such that resistance to cooling fluid flow in the caudal region of the nasal cavity is increased;
where the device is configured to pump fluid into the nasal cavity of the subject to cool the brain of the subject.

151. The cooling device of claim 145, where the second portion of the first nasal insert is configured to be disposed between the septum and an inferior nasal turbinate of the subject.

152. The cooling device of claim 150, where the second portion of the first nasal insert and the second portion of the second nasal insert is each configured to be disposed between the septum and an inferior nasal turbinate of the subject.

153. The cooling device of claim 145, where the second portion of the first nasal insert is configured to be disposed between the septum and a middle nasal turbinate of the subject.

154. The cooling device of claim 150, where the second portion of the first nasal insert and the second portion of the second nasal insert is each configured to be disposed between the septum and a middle nasal turbinate of the subject.

155.-165. (canceled)

166. The cooling device of claim 165, where the first nasal insert and the second nasal insert each comprises a pressure sensor.

167.-260. (canceled)

261. A cooling device comprising:
a pump;
a first tube configured to be inserted into a nostril of a subject and configured to terminate prior to the nasal valve such that fluid is directed into the nose, the first tube coupled to the pump such that the pump can pump fluid through the first tube and into the nose; and
a stopper configured to substantially prevent fluid from exiting the nose;
where the device is configured to pump fluid into the nasal cavity of the subject to cool the brain of the subject.

262. The cooling device of claim 261, further comprising:
a second tube configured to be inserted into a nostril of a subject and configured to terminate prior to the nasal valve such that fluid is directed into the nose, the second tube coupled to the pump such that the pump can pump fluid through the second tube and into the nose.

263. The cooling device of claim 262, where the pump comprises at least a first pump and a second pump, and the first tube is coupled to the first pump, and the second tube is coupled to the second pump.

264. The cooling device of claim 264, where the first tube and the second tube each comprises a pressure sensor.

265. A cooling device comprising:
a pump; and
a nasal mask configured to be placed about the nose of a subject such that fluid can enter the nostrils, the nasal mask coupled to the pump such that the pump can pump fluid into the nasal mask and into the nose of the subject;
where the device is configured to pump fluid into the nasal cavity of the subject to cool the brain of the subject.
